SEQUENCE LISTING (50 sequences)

SEQ ID NO: 1 - DNA, 2265 bp, Artificial Sequence
  Synthetic
  misc_feature (1)..(63): Leader Sequence
  misc_feature (64)..(201): CXCR4 domain
  misc_feature (202)..(336): CD8 hinge domain
  misc_feature (337)..(408): CD8 transmembrane domain
  misc_feature (409)..(1311): BMPRII domain
  misc_feature (1312)..(1347): Glycine linker
  misc_feature (1348)..(2262): ALK3 domain
atggcactgccagtgaccgccctgctgctgcctctggccctgctgctgcacgcagcaaggcctatggagggcatctccatctacacatctgacaattataccgaggagatgggctccggcgactacgattctatgaaggagccctgcttccgcgaggagaacgccaacttcaacaagattttcctgcctacaatctattccaccacaaccccagcaccccggccccctacacctgcaccaaccatcgccagccagcctctgtccctgaggccagaggcctgccgccccgcagcaggaggagcagtgcacacccgcggcctggactttgcctgcgacatctacatctgggcaccactggccggcacatgtggcgtgctgctgctgagcctggtcatcaccctgtattgtaagctgctggagctgatcggaaggggccgctacggagccgtgtataagggcagcctggacgagaggcctgtggcagtgaaggtgttctcctttgccaatcggcagaacttcatcaatgagaagaacatctacagagtgccactgatggagcacgataatatcgccaggtttatcgtgggcgacgagagggtgacagcagatggacgcatggagtacctgctggtcatggagtactatcccaacggcagcctgtgcaagtatctgtctctgcacaccagcgactgggtgagcagctgtaggctggcccactccgtgacacgcggcctggcctacctgcacaccgagctgccaagaggcgaccactataagcccgccatcagccaccgggacctgaacagccggaacgtgctggtgaagaatgacggcacatgcgtgatcagcgatttcggcctgtccatgaggctgaccggaaataggctggtgcgcccaggagaggaggataacgcagccatctctgaagtgggcacaatcagatacatggcaccagaggtgctggagggagcagtgaacctgagggactgcgagagcgccctgaagcaggtggatatgtacgccctgggcctgatctattgggagattttcatgagatgtaccgacctgtttcctggcgagtccgtgccagagtatcagatggccttccagacagaagtgggcaatcaccctacctttgaggatatgcaggtgctggtgtctcgggagaagcagagacccaagttccctgaggcctggaaggagaactctctggccgtgaggagcctgaaggagacaatcgaggactgctgggaccaggatgcagaggcaaggctgaccgcacagtgtgcagaggagaggatggcagagctgggaggaggctccggaggaggctctggcggcggcagcagaaccatcgccaagcagatccagatggtgcggcaagtgggcaagggcagatacggcgaagtgtggatgggcaagtggaggggcgagaaggtggccgtgaaggtgttctttacaaccgaggaggcctcctggttccgcgagacagaaatctaccagaccgtgctgatgcggcacgagaatatcctgggctttatcgccgccgacatcaagggcacaggctcttggacccagctgtacctgatcaccgattatcacgagaacggcagcctgtacgacttcctgaagtgcgccacactggatacccgcgccctgctgaagctggcctactccgccgcatgcggactgtgccacctgcacacagaaatctacggcacccagggcaagccagcaatcgcacaccgggacctgaagtccaagaatatcctgatcaagaagaacggctcttgctgtatcgccgatctgggcctggccgtgaagtttaatagcgacacaaacgaggtggatgtgcctctgaacacaagggtgggcaccaagcggtacatggccccagaggtgctggacgagtccctgaataagaaccacttccagccctacatcatggccgacatctactcttttggcctgatcatctgggagatggcccggagatgcatcaccggcggcatcgtggaggagtaccagctgccctactataatatggtgccatctgaccccagctatgaggatatgcgggaggtggtgtgcgtgaagcggctgagacctatcgtgtccaatcggtggaactctgacgagtgcctgagagccgtgctgaagctgatgtctgagtgttgggcacacaacccagccagccggctgacagccctgagaatcaagaagaccctggccaagatggtggagagccaggatgtgaagatttga

SEQ ID NO: 2 - protein, 754 aa, Artificial Sequence
  Synthesis
  MISC_FEATURE (1)..(21): CD8 Leader Sequence
  MISC_FEATURE (22)..(67): CXCR4 domain
  MISC_FEATURE (68)..(112): CD8 hinge domain
  MISC_FEATURE (113)..(136): CD8 transmembrane domain
  MISC_FEATURE (137)..(437): BMPRII domain
  MISC_FEATURE (438)..(449): Glycine linker
  MISC_FEATURE (450)..(754): ALK3 domain
MALPVTALLLPLALLLHAARPMEGISIYTSDNYTEEMGSGDYDSMKEPCFREENANFNKIFLPTIYSTTTPAPRPPTPAPTIASQPLSLRPEACRPAAGGAVHTRGLDFACDIYIWAPLAGTCGVLLLSLVITLYCKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELGGGSGGGSGGGSRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 3 - DNA, 2271 bp, Artificial Sequence
  Synthetic
  misc_feature (1)..(63): Leader Sequence
  misc_feature (64)..(207): CXCR4 domain
  misc_feature (208)..(342): CD8 hinge domain
  misc_feature (343)..(414): CD8 transmembrane domain
  misc_feature (415)..(1317): BMPRII domain
  misc_feature (1318)..(1353): Glycine linker
  misc_feature (1354)..(2268): ALK3 domain
atggcactgcctgtgacagccctgctgctgccactggccctgctgctgcacgcagccagacctatggaggacttcaacatggagtccgactctttcgaggatttttggaagggcgaggatctgagcaattactcctatagctccaccctgccaccttttctgctggacgcagcaccatgcgagccagagtccctggagatcaacaagaccacaacccctgcaccaagaccacccacaccagcacctaccatcgcctcccagccactgtctctgaggccagaggcctgtcgccctgcagcaggaggcgccgtgcacaccaggggcctggacttcgcctgcgacatctacatctgggcacccctggccggcacatgtggcgtgctgctgctgagcctggtcatcaccctgtattgcaagctgctggagctgatcggaaggggccgctacggagccgtgtataagggctctctggatgagaggcccgtggccgtgaaggtgttcagctttgccaaccggcagaatttcatcaacgagaagaatatctacagagtgcctctgatggagcacgacaacatcgccaggtttatcgtgggcgacgagagggtgacagcagatggacgcatggagtacctgctggtcatggagtactatcctaatggctctctgtgcaagtatctgagcctgcacacctccgattgggtgtctagctgtaggctggcccacagcgtgacacgcggcctggcctacctgcacaccgagctgcccagaggcgaccactataagcctgccatctctcaccgggatctgaacagcagaaatgtgctggtgaagaacgacggcacatgcgtgatcagcgatttcggcctgagcatgaggctgaccggaaacaggctggtgcgccctggagaggaggacaatgcagccatctccgaagtgggcacaatcagatacatggcaccagaggtgctggagggagcagtgaatctgagggactgcgagtctgccctgaagcaggtggatatgtacgccctgggcctgatctattgggagattttcatgcgctgtaccgatctgtttccaggcgagagcgtgcccgagtatcagatggccttccagacagaagtgggcaaccacccaacctttgaggacatgcaggtgctggtgtcccgggagaagcagagacctaagtttccagaggcctggaaggagaacagcctggccgtgcggagcctgaaggagacaatcgaggactgctgggaccaggatgcagaggcaaggctgaccgcacagtgtgcagaggagaggatggcagagctgggaggaggctctggaggaggcagcggaggcggctcccgcaccatcgccaagcagatccagatggtgcggcaagtgggcaagggcagatacggagaagtgtggatgggcaagtggaggggagagaaggtggcagtgaaggtgttctttacaaccgaggaggcctcctggttccgcgagacagaaatctaccagaccgtgctgatgcggcacgagaacatcctgggctttatcgccgccgacatcaagggcacaggctcctggacccagctgtacctgatcaccgattatcacgagaatggctctctgtacgacttcctgaagtgcgccacactggatacccgcgccctgctgaagctggcctactctgccgcatgcggactgtgccacctgcacacagaaatctacggcacccagggcaagcctgcaatcgcacaccgggatctgaagagcaagaacatcctgatcaagaagaatggctcctgctgtatcgccgacctgggcctggccgtgaagtttaacagcgacacaaatgaggtggatgtgccactgaacacaagggtgggcaccaagcggtacatggcccctgaggtgctggacgagagcctgaacaagaatcacttccagccatacatcatggccgacatctactcctttggcctgatcatctgggagatggcccggagatgcatcaccggcggcatcgtggaggagtaccagctgccatactataacatggtgccctctgaccctagctatgaggatatgcgggaggtggtgtgcgtgaagcggctgagacccatcgtgagcaaccggtggaacagcgatgagtgcctgagagccgtgctgaagctgatgtccgagtgttgggcccacaatcccgcctctcggctgacagccctgagaatcaagaagaccctggccaagatggtggagagccaggacgtgaagatttga

SEQ ID NO: 4 - protein, 756 aa, Artificial Sequence
  Synthesis
  MISC_FEATURE (1)..(21): CD8 Leader Sequence
  MISC_FEATURE (22)..(69): CXCR2 domain
  MISC_FEATURE (70)..(114): CD8 hinge domain
  MISC_FEATURE (115)..(138): CD8 transmembrane domain
  MISC_FEATURE (139)..(439): BMPRII domain
  MISC_FEATURE (440)..(451): Glycine linker
  MISC_FEATURE (452)..(756): ALK3 domain
MALPVTALLLPLALLLHAARPMEDFNMESDSFEDFWKGEDLSNYSYSSTLPPFLLDAAPCEPESLEINKTTTPAPRPPTPAPTIASQPLSLRPEACRPAAGGAVHTRGLDFACDIYIWAPLAGTCGVLLLSLVITLYCKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELGGGSGGGSGGGSRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 5 - DNA, 411 bp, Artificial Sequence
  Synthetic
  misc_feature (1)..(63): Leader Sequence
  misc_feature (64)..(201): CXCR4 domain
  misc_feature (202)..(336): CD8 hinge domain
  misc_feature (337)..(408): CD8 transmembrane domain
atggcactgccagtgacagccctgctgctgcctctggccctgctgctgcacgcagcaaggcctatggagggcatcagcatctacacatccgacaattataccgaggagatgggctccggcgactacgattctatgaaggagccatgcttcagggaggagaacgccaacttcaacaagattttcctgcccacaatctatagcaccacaaccccagcaccccgcccccctacacctgcaccaaccatcgcctctcagccactgagcctgaggcctgaggcctgcagaccagcagcaggaggagcagtgcacaccaggggcctggactttgcctgcgacatctacatctgggcacccctggccggcacatgtggcgtgctgctgctgagcctggtcatcaccctgtattgttga

SEQ ID NO: 6 - protein, 411 aa, Artificial Sequence
  Synthesis
  MISC_FEATURE (1)..(21): CD8 Leader Sequence
  MISC_FEATURE (22)..(67): CXCR4 domain
  MISC_FEATURE (68)..(112): CD8 hinge domain
  MISC_FEATURE (113)..(136): CD8 transmembrane domain
ATGGCACTGCCAGTGACAGCCCTGCTGCTGCCTCTGGCCCTGCTGCTGCACGCAGCAAGGCCTATGGAGGGCATCAGCATCTACACATCCGACAATTATACCGAGGAGATGGGCTCCGGCGACTACGATTCTATGAAGGAGCCATGCTTCAGGGAGGAGAACGCCAACTTCAACAAGATTTTCCTGCCCACAATCTATAGCACCACAACCCCAGCACCCCGCCCCCCTACACCTGCACCAACCATCGCCTCTCAGCCACTGAGCCTGAGGCCTGAGGCCTGCAGACCAGCAGCAGGAGGAGCAGTGCACACCAGGGGCCTGGACTTTGCCTGCGACATCTACATCTGGGCACCCCTGGCCGGCACATGTGGCGTGCTGCTGCTGAGCCTGGTCATCACCCTGTATTGTTGA

SEQ ID NO: 7 - DNA, 63 bp, Artificial Sequence
  Synthetic
atggcactgccagtgaccgccctgctgctgcctctggccctgctgctgcacgcagcaaggcct

SEQ ID NO: 8 - protein, 21 aa, Artificial Sequence
  Synthetic
MALPVTALLLPLALLLHAARP

SEQ ID NO: 9 - DNA, 138 bp, Artificial Sequence
  Synthetic
atggagggcatctccatctacacatctgacaattataccgaggagatgggctccggcgactacgattctatgaaggagccctgcttccgcgaggagaacgccaacttcaacaagattttcctgcctacaatctattcc

SEQ ID NO: 10 - protein, 46 aa, Artificial Sequence
  Synthetic
MEGISIYTSDNYTEEMGSGDYDSMKEPCFREENANFNKIFLPTIYS

SEQ ID NO: 11 - DNA, 145 bp, Artificial Sequence
  Synthetic
atggaggacttcaacatggagtccgactctttcgaggatttttggaagggcgaggatctgagcaattactcctatagctccaccctgccaccttttctgctggacgcagcaccatgcgagccagagtccctggagatcaacaaga

SEQ ID NO: 12 - protein, 48 aa, Artificial Sequence
  Synthetic
MEDFNMESDSFEDFWKGEDLSNYSYSSTLPPFLLDAAPCEPESLEINK

SEQ ID NO: 13 - DNA, 135 bp, Artificial Sequence
  Synthetic
accacaaccccagcaccccggccccctacacctgcaccaaccatcgccagccagcctctgtccctgaggccagaggcctgccgccccgcagcaggaggagcagtgcacacccgcggcctggactttgcctgcgac

SEQ ID NO: 14 - protein, 45 aa, Artificial Sequence
  Synthetic
TTTPAPRPPTPAPTIASQPLSLRPEACRPAAGGAVHTRGLDFACD

SEQ ID NO: 15 - DNA, 72 bp, Artificial Sequence
  Synthetic
atctacatctgggcaccactggccggcacatgtggcgtgctgctgctgagcctggtcatcaccctgtattgt

SEQ ID NO: 16 - protein, 24 aa, Artificial Sequence
  Synthetic
IYIWAPLAGTCGVLLLSLVITLYC

SEQ ID NO: 17 - DNA, 903 bp, Artificial Sequence
  Synthetic
aagctgctggagctgatcggaaggggccgctacggagccgtgtataagggcagcctggacgagaggcctgtggcagtgaaggtgttctcctttgccaatcggcagaacttcatcaatgagaagaacatctacagagtgccactgatggagcacgataatatcgccaggtttatcgtgggcgacgagagggtgacagcagatggacgcatggagtacctgctggtcatggagtactatcccaacggcagcctgtgcaagtatctgtctctgcacaccagcgactgggtgagcagctgtaggctggcccactccgtgacacgcggcctggcctacctgcacaccgagctgccaagaggcgaccactataagcccgccatcagccaccgggacctgaacagccggaacgtgctggtgaagaatgacggcacatgcgtgatcagcgatttcggcctgtccatgaggctgaccggaaataggctggtgcgcccaggagaggaggataacgcagccatctctgaagtgggcacaatcagatacatggcaccagaggtgctggagggagcagtgaacctgagggactgcgagagcgccctgaagcaggtggatatgtacgccctgggcctgatctattgggagattttcatgagatgtaccgacctgtttcctggcgagtccgtgccagagtatcagatggccttccagacagaagtgggcaatcaccctacctttgaggatatgcaggtgctggtgtctcgggagaagcagagacccaagttccctgaggcctggaaggagaactctctggccgtgaggagcctgaaggagacaatcgaggactgctgggaccaggatgcagaggcaaggctgaccgcacagtgtgcagaggagaggatggcagagctg

SEQ ID NO: 18 - protein, 301 aa, Artificial Sequence
  Synthetic
KLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAEL

SEQ ID NO: 19 - DNA, 37 bp, Artificial Sequence
  Synthetic
gggaggaggctccggaggaggctctggcggcggcagc

SEQ ID NO: 20 - protein, 12 aa, Artificial Sequence
  Synthetic
GGGSGGGSGGGS

SEQ ID NO: 21 - DNA, 918 bp, Artificial Sequence
  Synthetic
agaaccatcgccaagcagatccagatggtgcggcaagtgggcaagggcagatacggcgaagtgtggatgggcaagtggaggggcgagaaggtggccgtgaaggtgttctttacaaccgaggaggcctcctggttccgcgagacagaaatctaccagaccgtgctgatgcggcacgagaatatcctgggctttatcgccgccgacatcaagggcacaggctcttggacccagctgtacctgatcaccgattatcacgagaacggcagcctgtacgacttcctgaagtgcgccacactggatacccgcgccctgctgaagctggcctactccgccgcatgcggactgtgccacctgcacacagaaatctacggcacccagggcaagccagcaatcgcacaccgggacctgaagtccaagaatatcctgatcaagaagaacggctcttgctgtatcgccgatctgggcctggccgtgaagtttaatagcgacacaaacgaggtggatgtgcctctgaacacaagggtgggcaccaagcggtacatggccccagaggtgctggacgagtccctgaataagaaccacttccagccctacatcatggccgacatctactcttttggcctgatcatctgggagatggcccggagatgcatcaccggcggcatcgtggaggagtaccagctgccctactataatatggtgccatctgaccccagctatgaggatatgcgggaggtggtgtgcgtgaagcggctgagacctatcgtgtccaatcggtggaactctgacgagtgcctgagagccgtgctgaagctgatgtctgagtgttgggcacacaacccagccagccggctgacagccctgagaatcaagaagaccctggccaagatggtggagagccaggatgtgaagatttga

SEQ ID NO: 22 - protein, 305 aa, Artificial Sequence
  Synthetic
RTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 23 - DNA, 20 bp, Artificial Sequence
  Synthetic
caccggccaagtgtgcgtct

SEQ ID NO: 24 - DNA, 20 bp, Artificial Sequence
  Synthetic
gcagcgcgcacagctctttc

SEQ ID NO: 25 - DNA, 20 bp, Artificial Sequence
  Synthetic
cggcgaggaggaaggagcca

SEQ ID NO: 26 - DNA, 20 bp, Artificial Sequence
  Synthetic
tgggggcagaagacggaggg

SEQ ID NO: 27 - DNA, 20 bp, Artificial Sequence
  Synthetic
ggaggagaacaggaggaagg

SEQ ID NO: 28 - DNA, 22 bp, Artificial Sequence
  Synthetic
ctggatgtaacccccaaaatgc

SEQ ID NO: 29 - DNA, 20 bp, Artificial Sequence
  Synthetic
cgtgtggacaaggtggatga

SEQ ID NO: 30 - DNA, 19 bp, Artificial Sequence
  Synthetic
gccgcttaaacttgcctcg

SEQ ID NO: 31 - DNA, 20 bp, Artificial Sequence
  Synthetic
ggtgaagcaggcgtcggagg

SEQ ID NO: 32 - DNA, 20 bp, Artificial Sequence
  Synthetic
gagggcaatgccagccccag

SEQ ID NO: 33 - DNA, 18 bp, Artificial Sequence
  Synthetic
ccggaagcgaccctcaga

SEQ ID NO: 34 - DNA, 22 bp, Artificial Sequence
  Synthetic
cggtcaattttgccaaagatct

SEQ ID NO: 35 - DNA, 21 bp, Artificial Sequence
  Synthetic
ggactgcgcccatcacttacc

SEQ ID NO: 36 - DNA, 24 bp, Artificial Sequence
  Synthetic
aacttcatccaccggggctattac

SEQ ID NO: 37 - DNA, 21 bp, Artificial Sequence
  Synthetic
gccagagatgctgaagatggt

SEQ ID NO: 38 - DNA, 20 bp, Artificial Sequence
  Synthetic
gcaccaagttgggcatgaag

SEQ ID NO: 39 - DNA, 21 bp, Artificial Sequence
  Synthetic
tctctgctcctccctgttcta

SEQ ID NO: 40 - DNA, 20 bp, Artificial Sequence
  Synthetic
atgaaggggtcgttgatggc

SEQ ID NO: 41 - DNA, 20 bp, Artificial Sequence
  Synthetic
caccggccaagtgtgcgtct

SEQ ID NO: 42 - DNA, 20 bp, Artificial Sequence
  Synthetic
gcagcgcgcacagctctttc

SEQ ID NO: 43 - DNA, 20 bp, Artificial Sequence
  Synthetic
cggcgaggaggaaggagcca

SEQ ID NO: 44 - DNA, 20 bp, Artificial Sequence
  Synthetic
tgggggcagaagacggaggg

SEQ ID NO: 45 - DNA, 20 bp, Artificial Sequence
  Synthetic
ggaggagaacaggaggaagg

SEQ ID NO: 46 - DNA, 22 bp, Artificial Sequence
  Synthetic
ctggatgtaacccccaaaatgc

SEQ ID NO: 47 - DNA, 20 bp, Artificial Sequence
  Synthetic
cgtgtggacaaggtggatga

SEQ ID NO: 48 - DNA, 19 bp, Artificial Sequence
  Synthetic
gccgcttaaacttgcctcg

SEQ ID NO: 49 - DNA, 21 bp, Artificial Sequence
  Synthetic
acccactcctccacctttgac

SEQ ID NO: 50 - DNA, 22 bp, Artificial Sequence
  Synthetic
tgttgctgtagccaaattcgtt

(19) United States
(12) Patent Application Publication
(10) Pub. No.: US 2023/0302135 A1
(43) Pub. Date: Sep. 28, 2023

(54) HYBRID ALLOSTERIC RECEPTOR-ENGINEERED STEM CELLS

(71) Applicant: ARIZONA BOARD OF REGENTS ON BEHALF OF THE UNIVERSITY OF ARIZONA, Tucson, AZ (US)

(72) Inventors: Young-Wook WON, Tucson, AZ (US); Byeong-Hyun CHA, Tucson, AZ (US); David A. BULL, Tucson, AZ (US)

(21) Appl. No.: 18/042,263

(22) PCT Filed: Aug. 25, 2021
(86) PCT No.: PCT/US2021/047452

§ 371 Date: Feb. 20, 2023

Related U.S. Application Data
(60) Provisional application No. 63/070,427, filed on Aug. 26, 2020.

(51) Int. Cl. A61K 39/00; C07K 14/715; C07K 14/71; C07K 14/705; C12N 5/077

(52) U.S. Cl. CPC A61K 39/4637; C07K 14/7158; C07K 14/71; C07K 14/70517; C12N 5/0657; C07K 2319/03; C12N 2501/21; C12N 2501/06; C12N 2506/1353

(57) ABSTRACT

Described herein are mesenchymal stem cells (MCS) expressing hybrid allosteric receptors (HAR) that are responsive to stromal cell-derived factor 1 alpha (SDF-1α) secreted from acutely infarcted myocardium. Binding of SDF-1α to CXCR4 activates the co-stimulatory signals, bone morphogenetic protein 2 type II receptor (BMP2R2) and BMP type I receptor (ALK3), in order to accelerate the differentiation into cardiomyocytes. HAR-MSC CXCR4 differentiates into cardiomyocytes through Smad1/5 phosphorylation induced by the BMP2 signaling. In acute myocardial infarction (AMI) models, HAR-MSC CXCR4 treatment leads to the functional improvements by facilitated differentiation and increased cytokine secretion. HAR-MSC CXCR4 cells can be used for the treatment of AMI.

### CROSS-REFERENCE TO RELATED APPLICATIONS

This application claims priority to U.S. Provisional Patent Application No. 63/070,427 filed on Aug. 26, 2020, the contents of which is incorporated by reference herein in its entirety.

### FEDERALLY SPONSORED RESEARCH

This invention was made with government support under Grant No. HL138242, awarded by National Institutes of Health. The government has certain rights in the invention.

### REFERENCE TO SEQUENCE LISTING

This application is filed with a Computer Readable Form of a Sequence Listing in accord with 37 C.F.R § 1.821(c). The text file submitted by EFS, “212443-9010-WO01_sequence_listing_16-AUG-2021_ST25.txt,” was created on Aug. 16, 2021, contains 50 sequences, has a file size of 42.6 Kbytes, and is hereby incorporated by reference in its entirety.

### TECHNICAL FIELD

Described herein are mesenchymal stem cells (MCS) expressing hybrid allosteric receptors (HAR) that are responsive to stromal cell-derived factor 1 alpha (SDF-1α) secreted from acutely infarcted myocardiurn. The binding of SDF-1α to CXCR4 activates the co-stimulatory signals, bone morphogenetic protein 2 type II receptor (BMP2R2) and BMP type I receptor (ALK3), to induce differentiation into cardiomyocytes. HAR-MSC CXCR4 differentiates into cardiomyocytes through Smad1/5 phosphorylation induced by BMP2 signaling. In acute myocardial infarction (AMI) models, HAR-MSC CXCR4 treatment leads to functional improvements by facilitated differentiation and increased cytokine secretion. HAR-MSC CXCR4 cells can be used for the treatment of AMI.

### BACKGROUND

Acute myocardial infarction (AMI) is a leading cause of morbidity and mortality in the world. Coronary artery reperfusion and pharmacological treatment are the most successful options for patients in modern medicine; however, the outcome is contingent upon the progress of the disease and these treatments do not restore the coronary blood flow and the myocardial perfusion back to the pre-myocardial infarction (MI) status. There is no therapy available to cure the disease, prevent the disease progression, or reverse the ventricular failure. Stem cell-based regenerative therapies have shown promise for treating heart diseases including AMI.

Human mesenchymal stem cells (hMSCs) have shown promise in the field of regenerative medicine with their self-renewal capability and multi-lineage differentiation potential. The ease of isolation, the high migratory property, the relatively rapid expansion, and the low risk of graft versus host disease (GVHD) make MSCs the attractive candidates among various stem cells. There are still issues that need to be addressed. MSCs require prolonged culture periods to generate fully differentiated cells from hMSCs and this complicated process involves the use of various cytokines, growth factors, extracellular matrix molecules, and transcription factors. The resulting MSCs are often heterogeneous in their phenotypical characteristics, indicating the need for more accurate differentiation and culturing methods.

Chimeric antigen receptor (CAR) technology has emerged as a means for genetically modify T cells with synthetic receptors. The modified T cells are referred to as CAR-T cells and contain external antibody domains and internal co-stimulatory domains. Binding of the external antibody on CAR-T cells to its complimentary antigen on a target cancer cells stimulates the co-stimulatory domains to produce and secrete cancer-killing cytokines. This platform is applicable to the engineering of any cells; however, its applications are currently limited to cancer immunotherapies. Stem cell therapies for cardiovascular disease face challenges of inadequate homing and poor differentiation. CAR technology appears to have the potential for the modification of stem cells by (1) enabling targeting and (2) facilitating differentiation.

What is needed are stem cells expressing hybrid allosteric receptors (HAR) that can be used for the treatment of cardiovascular diseases including myocardial infarction.

### SUMMARY

One embodiment described herein is an engineered stem cell comprising a nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain. In one aspect, the HAR further comprises cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain. In another aspect, the stem cell is an embryonic stem cell, perinatal stem cell, adult stem cell, induced pluripotent stem cell, tissue-specific stem cell, mesenchymal stem cell, hematopoietic stem cell, mesenchymal stem cell, neural stem cell, or epithelial stem cell. In another aspect, the stem cell is a mesenchymal stem cell. In another aspect, the stem cell is a subject-derived stem cell. In another aspect, the stem cell is a mouse, rat, rabbit, pig, or human mesenchyrnal stem cell. In another aspect, the CXCR domain comprises a CXCR4 domain. In another aspect, the CXCR domain is encoded by a nucleic acid at least 90% identical to SEQ ID NO: 9. In another aspect, the CXCR domain comprises a polypeptide is at least 90% identical to SEQ ID NO: 10. In another aspect, the nucleic acid is at least 90% identical to SEQ ID NO: 1. In another aspect, the HAR the polypeptide is at least 90% identical to SEQ ID NO: 2. In another aspect, the nucleic acid encoding the HAR polypeptide is comprised in an extra chromosomal vector. In another aspect, the nucleic acid encoding the HAR polypeptide is integrated into the genome of the cell. In another aspect, expression of the HAR polypeptide is driven by a promoter.

Another embodiment described herein is a method or means for treating cardiovascular disease or disorder in a subject in need thereof, the method comprising contacting cardiovascular tissue with an effective amount of one or more engineered stem cells described herein, In one aspect, the cardiovascular disease or disorder comprises coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions. In another aspect, the cardiovascular disease or disorder is myocardial infarction.

Another embodiment described herein is a the use of an engineered stem cell described herein for treating or preventing a cardiovascular disease or disorder comprising coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions. In one aspect, the cardiovascular disease or disorder is myocardial infarction.

Another embodiment described herein is a nucleic acid encoding a hybrid allosteric receptor (HAR) polypeptide comprising an extracellular C-X-C chemokine receptor (CXCR) domain, an intracellular bone morphogenetic protein 2 type H receptor (BMP2RII) domain, and an intracellular bone morphogenetic protein (BMP) type 1 receptor (ALK3) domain. In one aspect, the HAR further comprises a duster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain. In another aspect, the nucleic acid is at least 90% identical to SEQ ID NO: 1 or the complement thereof. In another aspect, the nucleic acid encodes a HAR polypeptide at least 90% identical to SEQ ID NO: 2.

Another embodiment described herein is a an isolated a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain. In one aspect, the HAR further comprises a cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain. In another aspect, the polypeptide is encoded by a nucleic acid at least 90% identical to SEQ ID NO: 1. In another aspect, the HAR polypeptide is at least 90% identical to SEQ ID NO: 2.

Another embodiment described herein is a method for manufacturing an engineered stern cell, the method comprising: preparing a nucleic acid encoding a polypeptide comprising an extracellular C-X-C chemokine receptor (CXCR) domain, a cluster of differentiation 8 alpha (CD8α) hinge domain, a CD8α transmembrane domain, an intracellular bone morphogenetic protein 2 type II receptor (BMP2RII) domain, and an intracellular bone morphogenetic protein (BMP) type I receptor (ALK3) domain; introducing the nucleic acid of (a) into a stem cell; and isolating the transfected stem cells comprising the introduced nucleic acid. In one aspect, the nucleic acid is at least 90% identical to SEQ ID NO: 1. In another aspect, the stem cell is a mesenchymal stem cell. In another aspect, the stem cell expresses a HAR polypeptide.

Another embodiment described herein is an engineered stem cell produced by the methods described herein. In one aspect, the stem cell is a mesenchymal stem cell.

Another embodiment described herein is an engineered stem cell comprising a nucleic acid encoding a hybrid allosteric receptor (HAR) polypeptide comprising an extracellular receptor domain, and one or more intracellular domains. In one aspect, the extracellular domain and one or more intracellular domains are each selected from Chemokine (C-C motif) receptor 1 (CCR1), Chemokine (C-C motif) receptor 2 (CCR2), Chemokine (C-C motif) receptor 3 (CCR3), Chemokine (C-C motif) receptor 4 (CCR4), Chemokine (C-C motif) receptor 5 (CCR5), Chemokine (C-C motif) receptor 6 (CCR6), Chemokine (C-C motif) receptor 7 (CCR7), Chemokine (C-C motif) receptor 8 (CCR8), Chemokine (C-C motif) receptor 9 (CCR9), Chemokine (C-C motif) receptor 10 (CCR10), Chemokine (C-X-C motif) receptor 1 (CXCR1), Chemokine (C-X-C motif) receptor 2 (CXCR2), Chemokine (C-X-C motif) receptor 3 (CXCR3), Chemokine (C-X-C motif) receptor 4 (CXCR4), Chemokine (C-X-C motif) receptor 5 (CXCR5), Chemokine (C-X-C motif) receptor 6 (CXCR6), C-X3-C motif chemokine receptor 1 (CX3CR1), Chemokine (C motif) XC receptor 1 (XCR1), Atypical chemokine receptor 1 (ACKR1), Atypical chemokine receptor 2 (ACKR2), Atypical chemokine receptor 3 (ACKR3), Atypical chemokine receptor 4 (ACKR4), C-C Motif Chemokine Receptor Like 2 (CCRL2), Activin A Receptor Type 1 (ACVR1), Bone morphogenetic protein receptor type 1A (BMPRIA(ALK3)), Bone morphogenetic protein receptor type 1B (BMPRIB(ALK6)), Bone morphogenetic protein receptor type 2 (BMPR2), Activin A receptor type 2A (ACVR2A), Activin A receptor type 2B (ACVR2B), Transforming growth factor beta receptor 1 (TGFBR1), Transforming growth factor beta receptor 2 (TGFBR2), Vascular endothelial growth factor receptor 1 (VEGFR1), Vascular endothelial growth factor receptor 2 (VEGFR2), Vascular endothelial growth factor receptor 3 (VEGFR3), Epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), Platelet-derived growth factor receptor A (PDGFRα), Platelet-derived growth factor receptor B (PDGFRβ), Fibroblast growth factor receptor 1 (FGFR1), Fibroblast growth factor receptor 2 (FGFR2), Fibroblast growth factor receptor 3 (FGFR3), Fibroblast growth factor receptor 4 (FGFR4), Hepatocyte growth factor (HGF), Tropomyosin-related kinase A (TrkA), Tropomyosin-related kinase B (TrkB), Tropomyosin-related kinase C (TrkC), p75 neurotrophin receptor (p75NTR), Erythropoietin receptor (EPOR), Growth hormone receptor (GHR), Prolactin receptor (PRLR), Thrombopoietin receptor (TOPR), Granulocyte colony-stimulating factor receptor (GCSFR), Leukemia inhibitory factor receptor (LIFR), Ciliary neurotrophic factor receptor (CNTER), Cardiotrophin-like cytokine factor 1 (CLCF1), Oncostatin M receptor (OSMR), Interferon alpha receptor 1 (IFNAR1), Interferon alpha receptor 2 (IFNAR2), Glycoprotein 130 (gp130), Glycoprotein 140 (gp140), Integrin subunit alpha 1 (ITGA1), Integrin subunit alpha 2 (ITGA2), Integrin subunit alpha 3 (ITGA3), Integrin subunit alpha 4 (ITGA4), Integrin subunit alpha 5 (ITGA5), Integrin subunit alpha 6 (ITGA6), Integrin subunit alpha 7 (ITGA7), Integrin subunit alpha L (ITGAL), Integrin subunit alpha M (ITGAM), Integrin subunit alpha IIB (ITGAIIB), Integrin subunit alpha V (ITGAV), Integrin subunit beta 1 (ITGB1), Integrin subunit beta 2 (ITGB2), Integrin subunit beta 3 (ITGB3), Integrin subunit beta 4 (ITGB4), Integrin subunit beta 5 (ITGB5), Integrin subunit beta 6 (ITGB6), Integrin subunit beta 8 (ITGB8), Interleukin 1 receptor type 1 (IL1R1), Interleukin 1 receptor type 2 (IL1R2), Interleukin 2 receptor alpha (IL2RA), Interleukin 2 receptor beta (IL2RB), Interleukin 2 receptor gamma (IL2RG), Interleukin 3 receptor alpha (IDRA), Interleukin 4 receptor (IL4R), Interleukin 5 receptor alpha (IL5RA), Interleukin 6 receptor (IL6R), Interleukin 7 receptor (IL7R), Interleukin 9 receptor (IL9R), Interleukin 11 receptor alpha (IL11RA), Interleukin 12 receptor beta 1 (IL12RB1), Interleukin 13 receptor alpha 1 (IL13RA1), Interleukin 13 receptor alpha 2 (IL13RA2), Interleukin 15 receptor (IL15R), Interleukin 18 receptor 1 (IL18R1), Interleukin 21 receptor (IL21R), Interleukin 23 receptor (IL23R), Interleukin 27 receptor alpha (IL27RA), Interleukin 10 receptor alpha (IL10RA), Interleukin 10 receptor beta (IL10RB), Interleukin 20 receptor alpha (IL20RA), Interleukin 20 receptor beta (IL20RB), Interleukin 22 receptor alpha 1 (IL22RA1), Interleukin 22 receptor alpha 2 (IL22RA2), Interleukin 28 receptor alpha (IL28RA), or Interleukin 28 receptor beta (IL28RB). In another aspect, the HAR comprises one intracellular domain and two intracellular domains. In another aspect, the HAR further comprises a leader sequence, a hinge domain, a transmembrane domain between the extracellular domain and the first intracellular domain, and a linker between the two intracellular domains. In another aspect, the HAR comprises any of the domains listed in Table 3.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows schematic construct structures of the hybrid allosteric receptors (HARs) for BMP2Rs containing hCXCR4 HAR on human mesenchymal stem cells (hMSCs). Schematic representation of anti-hCXCR4-based HAR constructs containing the hCXCR4 extracellular ligand-binding domain alone (hCXCR4/Blank-HAR) or in combination with BMP2Rs intracellular domain (hCXCR4/BMP2Rs-HAR). A hCXCR2/BMP2Rs-HAR was also constructed and used as an antigen specific control. TM indicates the transmembrane region of CD8α.

FIGS. 2A-2B collectively show the transfection of hBMMSCs using pDNA/Ip/HA ternary complex. FIG. 2A shows a schematic depicting the procedure for the preparation for pDNA/Ip/HA ternary complex. FIG. 2B shows the results of luciferase reporter assays for hBMMSCs using luciferase-expressing plasmid DNA (pLuc)/Ip/HA ternary complex. n=3, mean±SD, ns, not significant, ***P<0.001 and ****P<0.0001 (two-way ANOVA). All data are representative of three independent experiments.

FIG. 3 shows the optimization of transfection on hCXCR4/BMP2Rs-HAR-mediated hMSCs using pDNA/Ip/HA ternary complex via hCXCR4 protein expression by Flow cytometry. All data are representative of three independent experiments.

FIGS. 4A-4G collectively show the transfection and expression of BMP2Rs containing hCXCR4 hybrid allosteric receptors (HARs) on hMSCs. FIG. 4A shows a schematic depicting the procedure for transfection and membrane localization by hCXCR4/BMP2Rs-HAR on hMSCs. FIG. 4B shows bright field and fluorescence images of actual binding of hCXCR4/BMP2Rs-HAR-engineered MSCs to FITC-labeled SDF-1 protein. Scale bars: 100 μm. FIG. 4C shows flow cytometry analysis of binding of hCXCR4/BMP2Rs-HAR-engineered MSCs to FITC-labeled SDF-1 protein. FIG. 4D shows immunofluorescence staining of BMP2RII (green) and ALK3 (BMP2RI, red) in all groups with SDF-1 treatment. Scale bars: 100 μm. FIG. 4E shows flow cytometry analysis of ALK3(BMP2RI) in all groups with SDF-1 treatment. FIG. 4F shows immunofluorescence staining of p-Smad1/5 (green) in all groups with SDF-1 treatment. White indicates the nuclear localization of p-Smad1/5. Scale bars: 25 μm. FIG. 4G shows flow cytometry analysis of p-Smad1/5 in all groups with SDF-1 treatment. Data are representative of three independent experiments.

FIGS. 5A-5C collectively show BMP signal expression via hCXCR2-HAR bound to IL-8. FIG. 5A shows a schematic depicting the overall procedure of BMP2 signal expression via hCXCR2-HAR bound to IL-8. FIG. 5B shows immunofluorescence staining of BMP2RII (green) and ALK3 (BMP2RI, red) in all groups with IL-8 treatment. FIG. 5C shows immunofluorescence staining of p-Smad1/5 (green) in all groups with IL-8 treatment. Scale bars: 50 μm. Data are representative of three independent experiments.

FIGS. 6A-6C collectively show in vitro cardiomyocyte differentiation of hMSCs expressing hCXCR4/BMP2Rs-HARs. FIG. 6A shows immunofluorescence staining of cTnT (green) and cTnI (red) in all groups with 5-aza and SDF-1 treatment. Scale bars: 50 μm. FIG. 6B shows flow cytometry analysis of cTnT and cTnI in all groups with 5-aza and SDF-1 treatment, FIG. 6C shows the gPCR analysis of relative mRNA expression of cardiac-specific marker (Nkx2.5, GATA4, cTnT, and cTnI) in all different groups with 5-aza and SDF-1 treatment. n=3, mean±SD, *P<0.05 (one-way ANOVA). Data are representative of three independent experiments.

FIGS. 7A-7D collectively show in vivo cardiac functional improvement of hMSCs expressing hCXCR4/BMP2Rs-HARs. FIG. 7A shows a schematic depicting the overall procedure of the administration of HAR-engineered hMSCs into AMI-induced rat hearts. FIG. 7B shows echocardiography-based quantification of various cardiac functional improvement-related categories in rat hearts in different groups 3 weeks post AMI. n=7, animal per groups, mean±SD, ns, not significant, *P<0.05, **P<0.01, and ***P<0.001 (one-way ANOVA). FIG. 7C shows representative photographs of Masson trichrome-staining (upper: red indicates viable myocardium; blue indicates collagen), anti-CD31 staining (middle: brown indicates blood vessels or the area of new capillary formation), and anti-cTnT staining using fast red chromogen (bottom: red indicates cTnT-expressed cardiac muscle cells) of paraffin sections of the ischemic region from the different groups. Scale bars: 150 μm. FIG. 7D shows the gPCR analysis of relative mRNA expression of rat angiogenesis (vWF and Pecam-1) and cardiomyocyte (cTnT) markers in all different groups. n=3, mean±SD, ***P<0.001 (one-way ANOVA). Data are representative of three independent experiments.

FIG. 8 shows the confirmation of in vivo cardiac functional improvement by hMSCs injection into rat infracted heart. (Left) The gPCR analysis of relative mRNA expression of human cardiomyocyte (cTnT) markers in different groups. n=3, mean±SD, ***P<0.001 (one-way ANOVA). (Right) The gPCR analysis of CT value ratio of rat/human GAPDH in different groups. n=3, mean±SD, ns, not significant, *P<0.05, and **P<0.01 (one-way ANOVA). Data are representative of three independent experiments.

FIG. 9 shows the cytokine-secretion profile of HAR-MSCs in the presence or absence of SDF-1, demonstrating increased production of cytokines associated with cardiogenic differentiation (IL-6 and LIF) and potential cardiac healing (anti-inflammatory, growth factor-, and cardiac repair-related chemokines) in the HAR-MSCs (CXCR4-BMP2RII/ALK3) exposed to SDF-1.

FIG. 10 shows SPECT images of ^{99m}_{Tc-}3P-RGD₂ in a rat ischemic heart, demonstrating robust angiogenesis in the myocardium 1-4 weeks post-infarction. Radioactive uptake in the LV wall is outlined by dashed lines.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present document, including definitions, will control. Preferred methods and materials are described below, although methods and materials similar or equivalent to those described herein can be used in practice or testing of the present invention.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a human. In another embodiment, the subject is a rat, mouse, or pig.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein “hybrid allosteric receptor” or “HAR” refers to an engineered artificial protein receptor that provides a cell the ability to target a specific tissue type and differentiate into a specific cell type. The HARs described herein comprise one or more C-X-C chemokine receptor (CXCR) domain and (2) one or more internal co-stimulatory domains of a bone morphogenetic protein receptors. In one exemplary embodiment described herein, HARs comprise extracellular CXCR domains and intracellular bone morphogenetic protein 2 type II receptor (BMP2R2) and a BMP type I receptor (ALK3) signaling domains, among other structural domains such as leader sequences, hinge domains, transrnernbrane domains, and linkers. The CXCR domain functions as a receptor for chemokine stromal cell-derived factor-1 (SDF-1) and the internal BMP2R2 and ALK3 domains permit differentiation-induction of the stem cells into cardiornyocytes through the phosphorylation of Smad transcriptional activators.

As used herein “stem cell” refers to an undifferentiated cell defined by its ability to self-renew and differentiate to produce progeny cells, including self-renewing progenitors, non-renewing progenitors, and terminally differentiated cells. Stem cells are characterized by their ability to differentiate into functional cells of various cell lineages from multiple germ layers (endoderm, mesoderm and ectoderm), as well as to give rise to tissues of multiple germ layers following transplantation and to contribute substantially to most, if not all, tissues following injection into blastocysts. Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multipotent stem cells; or (5) unipotent, meaning able to give rise to a single cell lineage (e.g., spermatogenic stem cells). Exemplary stem cells include embryonic stem cells, perinatal stem cells, adult stem cells, induced pluripotent stem cells, tissue-specific stem cells, mesenchymal stem cells, hematopoietic stem cells, mesenchymal stem cells, neural stem cells, or epithelial stem cells. In one aspect, the stem cell is a mesenchymal stem cell. In another aspect, the stem cell is an established line of mesenchymal stem cell. In another aspect, the stem cell is a subject-derived mesenchymal stem cell.

As used herein, the phrase “induced pluripotent stem cell (iPSC)” (or embryonic-like stem cell) refers to a proliferative and pluripotent stem cell which is obtained by de-differentiation of a somatic cell (e.g., an adult somatic cell).

As used herein “differentiation” refers to the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, “de-differentiation” refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein “cardiovascular disease or disorder” refers to any disease or disorder affecting the vascular system, including the heart and blood vessels. A cardiovascular disease or disorder includes any disease or disorder characterized by coronary or vascular dysfunction, including but not limited to coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions.

HAR technology was used to design HAR-MSCs to induce MSCs to migrate toward infarcted myocardium followed by rapid differentiation into cardiomyocytes. The HAR construct comprises (1) the external C-X-C chemokine receptor 4 (CXCR4) and (2) internal co-stimulatory domains of a bone morphogenetic protein 2 type H receptor (BMP2R2) and a BMP type I receptor (ALK3). In HAR-MSC CXCR4, CXCR4 serves as a receptor for chemokine stromal cell-derived factor-1 (SDF-1) and the internal BMP2R2 and ALK3 domains are for inducing cardiac differentiation of the hMSCs. SDF-1 is up-regulated in myocardium immediately upon AMI, binds to HAR-MSC CXCR4 cells, and this binding stimulates the BMP2R2 and ALK3 pathways. The interaction of ALK3 with BMP2R2 then induces the differentiation of MSCs into cardiomyocytes through the phosphorylation of Smad transcriptional activators.

HAR-MSCs CXCR4 has shown facilitated cardiomyocyte differentiation in vitro upon binding of SDF-1 and this enriched differentiation involves the phosphorylation of Smad 1/5 initiated by the BMP2 signaling. HAR-MSC CXCR4 later revealed the characteristics similar to cardiornyocytes. The functional improvement in rat models of AMI upon direct injection of HAR-MSC CXCR4 has been verified. Consequently, HAR-MSC CXCR4 has proven its potential as a treatment for cardiovascular disease and a novel methodology for stem cell engineering.

One embodiment described herein is an engineered stem cell comprising a nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain. In one aspect, the HAR further comprises cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain. In another aspect, the stem cell is an embryonic stem cell, perinatal stem cell, adult stem cell, induced pluripotent stem cell, tissue-specific stem cell, mesenchymal stem cell, hematopoietic stem cell, mesenchymal stem cell, neural stem cell, or epithelial stem cell. In another aspect, the stem cell is a mesenchymal stem cell. In another aspect, the stem cell is a subject-derived stem cell. In another aspect, the stem cell is a mouse, rat, rabbit, pig, or human mesenchymal stem cell. In another aspect, the CXCR domain comprises a CXCR4 domain. In another aspect, the CXCR domain is encoded by a nucleic acid at least 90% identical to SEQ ID NO: 9. In another aspect, the CXCR domain comprises a polypeptide is at least 90% identical to SEQ ID NO: 10. In another aspect, the nucleic acid is at least 90% identical to SEQ ID NO: 1. In another aspect, the HAR the polypeptide is at least 90% identical to SEQ ID NO: 2. In another aspect, the nucleic acid encoding the HAR polypeptide is comprised in an extra chromosomal vector. In another aspect, the nucleic acid encoding the HAR polypeptide is integrated into the genome of the cell, In another aspect, expression of the HAR polypeptide is driven by a promotor.

Another embodiment described herein is a method or means for treating cardiovascular disease or disorder in a subject in need thereof, the method comprising contacting cardiovascular tissue with an effective amount of one or more engineered stem cells described herein, In one aspect, the cardiovascular disease or disorder comprises coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions. In another aspect, the cardiovascular disease or disorder is myocardial infarction.

Another embodiment described herein is a the use of an engineered stem cell described herein for treating or preventing a cardiovascular disease or disorder comprising coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions. In one aspect, the cardiovascular disease or disorder is myocardial infarction.

Another embodiment described herein is a nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain. In one aspect, the HAR further comprises a cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain. In another aspect, the nucleic acid is at least 90% identical to SEQ ID NO: 1 or the complement thereof. In another aspect, the nucleic acid encodes a HAR polypeptide at least 90% identical to SEQ ID NO: 2.

Another embodiment described herein is a an isolated a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type receptor (ALK3) domain. In one aspect, the HAR further comprises a cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain. In another aspect, the polypeptide is encoded by a nucleic acid at least 90% identical to SEQ ID NO: 1. In another aspect; the HAR polypeptide is at least 90% identical to SEQ ID NO: 2.

Another embodiment described herein is a method for manufacturing an engineered stem cell, the method comprising: preparing a nucleic acid encoding a polypeptide comprising an extracellular C-X-C chemokine receptor (CXCR) domain, a cluster of differentiation 8 alpha (CD8α) hinge domain, a CD8α transmembrane domain, an intracellular bone morphogenetic protein 2 type II receptor (BMP2RII) domain, and an intracellular bone morphogenetic protein (BMP) type 1 receptor (ALK3) domain; introducing the nucleic acid of (a) into a stem cell; and isolating the transfected stem cells comprising the introduced nucleic acid. In one aspect, the nucleic acid is at least 90% identical to SEQ ID NO: 1. In another aspect, the stem cell is a mesenchymal stem cell. In another aspect, the stem cell expresses a HAR polypeptide.

Another embodiment described herein is an engineered stem cell produced by the methods described herein. In one aspect, the stem cell is a mesenchymal stem cell.

Another embodiment described herein is an engineered stem cell comprising a nucleic acid encoding a hybrid allosteric receptor (HAR) polypeptide comprising an extracellular receptor domain, and one or more intracellular domains. In one aspect; the extracellular domain and one or more intracellular domains are each selected from Chemokine (C-C motif) receptor 1 (CCR1), Chemokine (C-C motif) receptor 2 (CCR2), Chemokine (C-C motif) receptor 3 (CCR3), Chemokine (C-C motif) receptor 4 (CCR4), Chemokine (C-C motif) receptor 5 (CCR5), Chemokine (C-C motif) receptor 6 (CCR6), Chemokine (C-C motif) receptor 7 (CCR7), Chemokine (C-C motif) receptor 8 (CCR8), Chemokine (C-C motif) receptor 9 (CCR9), Chemokine (C-C motif) receptor 10 (CCR10), Chemokine (C-X-C motif) receptor 1 (CXCR1), Chemokine (C-X-C motif) receptor 2 (CXCR2), Chemokine (C-X-C motif) receptor 3 (CXCR3), Chemokine (C-X-C motif) receptor 4 (CXCR4), Chemokine (C-X-C motif) receptor 5 (CXCR5), Chemokine (C-X-C motif) receptor 6 (CXCR6), C-X3-C motif chemokine receptor 1 (CX3CR1), Chemokine (C motif) XC receptor 1 (XCR1), Atypical chemokine receptor 1 (ACKR1), Atypical chemokine receptor 2 (ACKR2), Atypical chemokine receptor 3 (ACKR3), Atypical chemokine receptor 4 (ACKR4), C-C Motif Chemokine Receptor Like 2 (CCRL2), Activin A Receptor Type 1 (ACVR1), Bone morphogenetic protein receptor type 1A (BMPRIA(ALK3)), Bone morphogenetic protein receptor type 1B (BMPRIB(ALK6)), Bone morphogenetic protein receptor type 2 (BMPR2), Activin A receptor type 2A (ACVR2A), Activin A receptor type 2B (ACVR2B), Transforming growth factor beta receptor 1 (TGFBR1), Transforming growth factor beta receptor 2 (TGFBR2), Vascular endothelial growth factor receptor 1 (VEGFR1), Vascular endothelial growth factor receptor (VEGFR2), Vascular endothelial growth factor receptor 3 (VEGFR3), Epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), Platelet-derived growth factor receptor A (PDGFRα), Platelet-derived growth factor receptor B (PDGFRβ), Fibroblast growth factor receptor 1 (FGFR1), Fibroblast growth factor receptor 2 (FGFR2), Fibroblast growth factor receptor 3 (FGFR3), Fibroblast growth factor receptor 4 (FGFR4), Hepatocyte growth factor (HGF), Tropomyosin-related kinase A (TrkA), Tropomyosin-related kinase B (TrkB), Tropomyosin-related kinase C (TrkC), p75 neurotrophin receptor (p75NTR), Erythropoietin receptor (EPOR), Growth hormone receptor (GHR), Prolactin receptor (PRLR), Thrombopoietin receptor (TOPR), Granulocyte colony-stimulating factor receptor (GCSFR), Leukemia inhibitory factor receptor (LIFR), Ciliary neurotrophic factor receptor (CNTER), Cardiotrophin-like cytokine factor 1 (CLCF1), Oncostatin M receptor (OSMR), Interferon alpha receptor 1 (IFNAR1), Interferon alpha receptor 2 (IFNAR2), Glycoprotein 130 (gp130), Glycoprotein 140 (gp140), Integrin subunit alpha 1 (ITGA1), Integrin subunit alpha 2 (ITGA2), Integrin subunit alpha 3 (ITGA3), Integrin subunit alpha 4 (ITGA4), Integrin subunit alpha 5 (ITGA5), Integrin subunit alpha 6 (ITGA6), Integrin subunit alpha 7 (ITGA7), Integrin subunit alpha L (ITGAL), Integrin subunit alpha M (ITGAM), Integrin subunit alpha IIB (ITGAIIB), Integrin subunit alpha V (ITGAV), Integrin subunit beta 1 (ITGB1), Integrin subunit beta 2 (ITGB2), Integrin subunit beta 3 (ITGB3), Integrin subunit beta 4 (ITGB4), Integrin subunit beta 5 (ITGB5), Integrin subunit beta 6 (ITGB6), Integrin subunit beta 8 (ITGB8), Interleukin 1 receptor type 1 (IL1R1), Interleukin 1 receptor type 2 (IL1R2), Interleukin 2 receptor alpha (IL2RA), Interleukin 2 receptor beta (IL2RB), Interleukin 2 receptor gamma (IL2RG), Interleukin 3 receptor alpha (IL3RA), Interleukin 4 receptor (IL4R), Interleukin 5 receptor alpha (IL5RA), Interleukin 6 receptor (IL6R), Interleukin 7 receptor (IL7R), Interleukin 9 receptor (IL9R), Interleukin 11 receptor alpha (IL11RA), Interleukin 12 receptor beta 1 (IL12RB1), Interleukin 13 receptor alpha 1 (IL13RA1), Interleukin 13 receptor alpha 2 (IL13RA2), Interleukin 15 receptor (IL15R), Interleukin 18 receptor 1 (IL18R1), Interleukin 21 receptor (IL21R), Interleukin 23 receptor (IL23R), Interleukin 27 receptor alpha (IL27RA), Interleukin 10 receptor alpha (IL10RA), Interleukin 10 receptor beta (IL10RB), Interleukin 20 receptor alpha (IL20RA), Interleukin 20 receptor beta (IL20RB), Interleukin 22 receptor alpha 1 (IL22RA1), Interleukin 22 receptor alpha 2 (IL22RA2), Interleukin 28 receptor alpha (IL28RA), or Interleukin 28 receptor beta (IL28RB). In another aspect, the NAR comprises one intracellular domain and two intracellular domains. In another aspect, the HAR further comprises a leader sequence, a hinge domain, a transmembrane domain between the extracellular domain and the first intracellular domain, and a linker between the two intracellular domains. In another aspect, the HAR comprises any of the domains listed in Table 3.

Another embodiment described herein is a polynucleotide vector comprising one or more nucleotide sequences described herein.

Another embodiment described herein is a cell comprising one or more nucleotide sequences described herein or a polynucleotide vector described herein. In one aspect, the cell is a stem cell, In another aspect, the cell is a mesenchymal stem cell. In another aspect, the stem cell is isolated from a subject in need of treatment.

Another embodiment is a nucleotide sequence described herein. In one aspect, the nucleotide sequence has 85% to 99% identity to SEQ ID NO: 1, 3, 5, 7, 9, 11, 13, 15, 17, 19, or 21. In another aspect, the polypeptide is selected from SEQ ID NO: 1, 3, 5, 7, 9, 11, 13, 15, 17, 19, or 21.

Another embodiment is a polypeptide encoded by a nucleotide sequence described herein. In one aspect, the polypeptide has 85% to 99% identity to SEQ ID NO: 2, 4, 6, 8, 10, 12, 14, 16, 18, 20, or 22. In another aspect, the polypeptide is selected from SEQ ID NO: 2, 4, 6, 8, 10, 12, 14, 16, 18, 20, or 22.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; and, optionally, isolating the polypeptide encoded by a nucleotide described herein.

Another embodiment described herein is a nucleotide sequence or a polypeptide encoded by the nucleotide sequence produced by the method or the means described herein

Another embodiment described herein is a method of treatment comprising administering an effective amount to a subject in need thereof of one or more stem cells comprising one or more nucleotide sequences described herein encoding one or more of the HAR polypeptides described herein.

Another embodiment described herein is the use of an effective amount of one or more stem cells comprising one or more nucleotide sequences described herein for the treatment of a disease or disorder comprising a administering an effective amount of one or more stem cells comprising one or more nucleotide sequences described herein to a subject in need thereof. In one aspect, the disease or disorder is coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism; or other coronary or vascular conditions. In one aspect, the disease or disorder is myocardial infarction. In another aspect, the stem cell comprises one or more nucleotide sequences encoding one or more of the HAR polypeptides described herein.

Another embodiment described herein is a research tool comprising a polypeptide encoded by a nucleotide sequence described herein.

Another embodiment described herein is a reagent comprising a polypeptide encoded by a nucleotide sequence described herein.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% identical to (a) nucleotide sequences, or degenerate, homologous, or codon-optimized variants thereof, encoding polypeptides having the amino acid sequences in SEQ ID NOs: 1, 3, 5, 7, 9, 11, 13, 15, 17, 19, or 21; (b) nucleotide sequences, or degenerate, homologous, or codon-optimized variants thereof, encoding polypeptides having the amino acid sequences in SEQ ID NOs: 2, 4, 6, 8, 10, 12, 14, 16, 18, 20, or 22; and (c) nucleotide sequences capable of hybridizing to the complement of any of the nucleotide sequences in (a) or (b) above and capable of expressing functional polypeptides of amino acid sequences in SEQ ID NOs: 2, 4, 6, 8, 10, 12, 14, 16, 18, 20, or 22.

By a polynucleotide having a nucleotide sequence at least, for example, 90-99% “identical” to a reference nucleotide sequence encoding HAR is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the HAR.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence, or deleted therefrom. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence. A sequence identical to a reference sequence has 100% identity to the reference sequence or is that sequence.

A sequence can comprises a sequence identical to a specific Sequence Identification Number (SEQ ID NO:) or having a particular percent identity to a SEQ ID NO. A sequence comprising a sequence provided as a SEQ ID NO can contain additional residues at either termini. Further a particular SEQ ID may comprise multiple sequences that are also listed in individual SEQ ID Nos. For example, a SEQ ID NO for a contiguous HAR construct (e.g., SEQ ID NO: 1 or SEQ ID NO: 2), may comprise multiple SEQ ID Nos for the individual domains of the construct (e.g., SEQ ID NO: 7, 9, 13, 17, 19, and 21; or SEQ ID NO: 8, 10, 14, 16, 18, 20, and 22, respectively). These individual domains can be reconfigured in various orders and multiples to create alternative constructs which are envisioned within the scope of the description herein,

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res, 14(6): 6745-6763 (1986). An implementation of this algorithm for nucleic acid and peptide sequences is provided by the Genetics Computer Group (Madison, Ws.) in their BESTFIT utility application. Alternatively, the Clustal Omega (EMBL-EBI) can be used for pairwise or multiple alignments of nucleic acids or polypeptides. Maderia et al., Nucl. Acid Res. 47(W1): W636-W641 (2019).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NOs: 1, 3, 5, 7, 9, 11, 13, 15, 17, 19, or 21, or degenerate, homologous, or codon-optimized variants thereof, will encode a FEAR or subdomains thereof.

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247: 1306-1310 (1990), wherein the authors indicate that proteins are surprisingly tolerant of amino acid substitutions. Thus, fragments, derivatives, or analogs of the polypeptides of SEQ ID NOs: 2, 4, 6, 8, 10, 12, 14, 16, 18, 20, or 22 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue.). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an fusion protein, leader sequence, secretory sequence, or a sequence which is employed for purification of the mature polypeptide or a proprotein sequence.

In addition, fragments, derivatives, or analogs of the polypeptides of SEQ ID NOs: 2, 4, 6, 8, 10, 12, 14, 16, 18, 20, or 22 can be substituted with one or more conserved or non-conserved amino acid residue (preferably a conserved amino acid residue). In some cases these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99%, or 100% identical to the polypeptide sequence shown in SEQ ID NOs: 2, 4, 6, 8, 10, 12, 14, 16, 18, 20, or 22 and will comprise functional or non-functional proteins. Similarly, additions or deletions to the polypeptides can be made either at the N- or C-termini or within non-conserved regions of the polypeptide or to particular non-conserved amino acids (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The compositions, formulations, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

Various embodiments and aspects of the inventions described herein are summarized by the following clauses:
Clause 1. An engineered stem cell comprising a nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain.
Clause 2. The stem cell of clause 1, wherein the HAR further comprises cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain.
Clause 3. The stem cell of clause 1 or 2, wherein the stem cell is an embryonic stem cell, perinatal stem cell, adult stem cell, induced pluripotent stem cell, tissue-specific stem cell, mesenchymal stem cell, hematopoietic stem cell, mesenchymal stem cell, neural stem cell, or epithelial stem cell.
Clause 4. The stem cell of any one of clauses 1-3, wherein the stem cell is a mesenchymal stem cell.
Clause 5. The stem cell of any one of clauses 1-4, wherein the stem cell is a subject-derived stem cell.
Clause 6. The stem cell of any one of clauses 1-5, wherein the stem cell is a mouse, rat, rabbit, pig, or human mesenchymal stem cell.
Clause 7. The stem cell of any one of clauses 1-6, wherein the CXCR domain comprises a CXCR4 domain.
Clause 8. The stem cell of any one of clauses 1-7, wherein the CXCR domain is encoded by a nucleic acid at least 90% identical to SEQ ID NO: 9.
Clause 9. The stem cell of any one of clauses 1-8, wherein the CXCR domain comprises a polypeptide is at least 90% identical to SEQ ID NO: 10.
Clause 10. The stem cell of any one of clauses 1-9, wherein the nucleic acid is at least 90% identical to SEQ ID NO: 1.
Clause 11, The stem cell of any one of clauses 1-10, wherein the HAR the polypeptide is at least 90% identical to SEQ ID NO: 2.
Clause 12. The stem cell of any one of clauses 1-11, wherein the nucleic acid encoding the HAR polypeptide is comprised in an extra chromosomal vector.
Clause 13. The stem cell of any one of clauses 1-12, wherein the nucleic acid encoding the HAR polypeptide is integrated into the genome of the cell.
Clause 14. The stem cell of any one of clauses 1-13, wherein expression of the HAR polypeptide is driven by a promotor.
Clause 15. A method or means for treating cardiovascular disease or disorder in a subject in need thereof, the method comprising contacting cardiovascular tissue with an effective amount of one or more of the engineered stem cells of any one of clauses 1-14.
Clause 16. The method of clause 15, wherein the cardiovascular disease or disorder comprises coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions.
Clause 17. The method of clause 15 or 16, wherein the cardiovascular disease or disorder is myocardial infarction.
Clause 18. Use of an engineered stem cell of any one of clauses 1-14 for treating or preventing a cardiovascular disease or disorder comprising coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions.
Clause 19. The use of clause 18, wherein the cardiovascular disease or disorder is myocardial infarction.
Clause 20. A nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain.
Clause 21. The nucleic acid of clause 20, wherein the HAR further comprises a duster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain.
Clause 22. The nucleic acid of clause 20 or 21, wherein the nucleic acid is at least 90% identical to SEQ ID NO: 1 or the complement thereof.
Clause 23. The nucleic acid of any one of clauses 20-22, wherein the nucleic acid encodes a HAR polypeptide at least 90% identical to SEQ ID NO: 2.
Clause 24. An isolated a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type 1 receptor (ALK3) domain.
Clause 25. The isolated HAR of clause 24, wherein the HAR further comprises a cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain.
Clause 26. The isolated HAR of clause 24 or 25, wherein the polypeptide is encoded by a nucleic acid at least 90% identical to SEQ ID NO: 1.
Clause 27. The isolated HAR of any one of clauses 24-26, wherein the HAR polypeptide is at least 90% identical to SEQ ID NO: 2.
Clause 28. A method for manufacturing an engineered stem cell, the method comprising:
   (a) preparing a nucleic acid encoding a polypeptide comprising an extracellular C-X-C chemokine receptor (CXCR) domain, a cluster of differentiation 8 alpha (CD8α) hinge domain, a CD8α transmembrane domain, an intracellular bone morphogenetic protein 2 type II receptor (BMP2RII) domain, and an intracellular bone morphogenetic protein (BMP) type I receptor (ALK3) domain;
   (b) introducing the nucleic acid of (a) into a stem cell; and
   (c) isolating the transfected stem cells comprising the introduced nucleic acid.
Clause 29. The method of clause 28, wherein the nucleic acid is at least 90% identical to SEQ ID NO: 1.
Clause 30. The method of clause 28 or 29, wherein the stem cell is a mesenchymal stem cell.
Clause 31. The method of any one of clauses 28-X, wherein the stem cell expresses a HAR polypeptide.
Clause 32. An engineered stem cell produced by the method of any one of clauses 28-X.
Clause 33. The stem cell of clause 32, wherein the stem cell is a mesenchymal stem cell.
Clause 34. An engineered stem cell comprising a nucleic acid encoding a hybrid allosteric receptor (LIAR) polypeptide comprising an extracellular receptor domain, and one or more intracellular domains.
Clause 35. The stem cell of clause 34, wherein the extracellular domain and one or more intracellular domains are each selected from Chemokine (C-C motif) receptor 1 (CCR1), Chemokine (C-C motif) receptor 2 (CCR2), Chemokine (C-C motif) receptor 3 (CCR3), Chemokine (C-C motif) receptor 4 (CCR4), Chemokine (C-C motif) receptor 5 (CCR5), Chemokine (C-C motif) receptor 6 (CCR5), Chemokine (C-C motif) receptor 7 (CCR7), Chemokine (C-C motif) receptor 8 (CCR3), Chemokine (C-C motif) receptor 9 (CCR9), Chemokine (C-C motif) receptor 10 (CCR10), Chemokine (C-X-C motif) receptor 1 (CXCR1), Chemokine (C-X-C motif) receptor 2 (CXCR2), Chemokine (C-X-C motif) receptor 3 (CXCR3), Chemokine (C-X-C motif) receptor 4 (CXCR4), Chemokine (C-X-C motif) receptor 5 (CXCR5), Chemokine (C-X-C motif) receptor 6 (CXCR6), C-X3-C motif chemokine receptor 1 (CX3CR1), Chemokine (C motif) XC receptor 1 (XCR1), Atypical chemokine receptor 1 (ACKR1), Atypical chemokine receptor 2 (ACKR2), Atypical chemokine receptor 3 (ACKR3), Atypical chemokine receptor 4 (ACKR4), C-C Motif Chemokine Receptor Like 2 (CCRL2), Activin A Receptor Type 1 (ACVR1), Bone morphogenetic protein receptor type 1A (BMPRIA(ALK3)), Bone morphogenetic protein receptor type 1B (BMPRIB(ALK6)), Bone morphogenetic protein receptor type 2 (BMPR2), Activin A receptor type 2A (ACVR2A), Activin A receptor type 2B (ACVR2B), Transforming growth factor beta receptor 1 (TGFBR1), Transforming growth factor beta receptor 2 (TGFBR2), Vascular endothelial growth factor receptor 1 (VEGFR1), Vascular endothelial growth factor receptor 2 (VEGFR2), Vascular endothelial growth factor receptor 3 (VEGFR3), Epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), Platelet-derived growth factor receptor A (PDGFRα), Platelet-derived growth factor receptor B (PDGFRβ), Fibroblast growth factor receptor 1 (FGFR1), Fibroblast growth factor receptor 2 (FGFR2), Fibroblast growth factor receptor 3 (FGFR3), Fibroblast growth factor receptor 4 (FGFR4), Hepatocyte growth factor (HGF), Tropomyosin-related kinase A (TrkA), Tropomyosin-related kinase B (TrkB), Tropomyosin-related kinase C (TrkC), p75 neurotrophin receptor (p75NTR), Erythropoietin receptor (EPOR), Growth hormone receptor (GHR), Prolactin receptor (PRLR), Thrombopoietin receptor (TOPR), Granulocyte colony-stimulating factor receptor (GCSFR), Leukemia inhibitory factor receptor (LIFR), Ciliary neurotrophic factor receptor (CNTER), Cardiotrophin-like cytokine factor 1 (CLCF1), Oncostatin M receptor (OSMR), Interferon alpha receptor 1 (IFNAR1), Interferon alpha receptor 2 (IFNAR2), Glycoprotein 130 (gp130), Glycoprotein 140 (gp140), Integrin subunit alpha 1 (ITGA1), Integrin subunit alpha 2 (ITGA2), Integrin subunit alpha 3 (ITGA3), Integrin subunit alpha 4 (ITGA4), Integrin subunit alpha 5 (ITGA5), Integrin subunit alpha 6 (ITGA6), Integrin subunit alpha 7 (ITGA7), Integrin subunit alpha L (ITGAL), Integrin subunit alpha M (ITGAM), Integrin subunit alpha IIB (ITGAIIB), Integrin subunit alpha V (ITGAV), Integrin subunit beta 1 (ITGB1), Integrin subunit beta 2 (ITGB2), Integrin subunit beta 3 (ITGB3), Integrin subunit beta 4 (ITGB4), Integrin subunit beta 5 (ITGB5), Integrin subunit beta 6 (ITGB6), Integrin subunit beta 8 (ITGB8), Interleukin 1 receptor type 1 (IL1R1), Interleukin 1 receptor type 2 (IL1R2), Interleukin 2 receptor alpha (IL2RA), Interleukin 2 receptor beta (IL2RB), Interleukin 2 receptor gamma (IL2RG), Interleukin 3 receptor alpha (IL3RA), Interleukin 4 receptor (IL4R), Interleukin 5 receptor alpha (IL5RA), Interleukin 6 receptor (IL6R), Interleukin 7 receptor (IL7R), Interleukin 9 receptor (IL9R), Interleukin 11 receptor alpha (IL11RA), Interleukin 12 receptor beta 1 (IL12RB1), Interleukin 13 receptor alpha 1 (IL13RA1), Interleukin 13 receptor alpha 2 (IL13RA2), Interleukin 15 receptor (IL15R), Interleukin 18 receptor 1 (IL18R1), Interleukin 21 receptor (IL21R), Interleukin 23 receptor (IL23R), Interleukin 27 receptor alpha (IL27RA), Interleukin 10 receptor alpha (IL10RA), Interleukin 10 receptor beta (IL10RB), Interleukin 20 receptor alpha (IL20RA), Interleukin 20 receptor beta (IL20RB), Interleukin 22 receptor alpha 1 (IL22RA1), Interleukin 22 receptor alpha 2 (IL22RA2), Interleukin 28 receptor alpha (IL28RA), or Interleukin 28 receptor beta (IL28RB).
Clause 36. The stem cell of clause 34 or 35, wherein the HAR comprises one intracellular domain and two intracellular domains.
Clause 37. The stem cell of any one of clauses 34-36, wherein the HAR further comprises a leader sequence, a hinge domain, a transmembrane domain between the extracellular domain and the first intracellular domain, and a linker between the two intracellular domains.
Clause 38. The stem cell of any one of clauses 34-37, wherein the HAR comprises any of the domains listed in Table 3.

### EXAMPLES

### Example 1

### Materials

All materials were purchased from either Sigma-Aldrich or Thermo Fisher Scientific and used as received unless otherwise noted.

### Cell Lines and Culture Conditions

Human bone marrow mesenchymal stem cells (hBMMSCs) were purchased from the American Type Cell Culture (ATCC). The cells were maintained in Dulbecco's Modified Eagle's (DMEM, Gibco Invitrogen), supplemented with 10% fetal bovine serum (FBS, Gibco Invitrogen), and 1% penicillin/streptomycin (P/S, Gibco Invitrogen) at 37° C. in a humidified incubator containing 95% air and 5% CO₂. The media was refreshed every 3 days. Cells and transfected cells, cultured to approximately 80% confluence, were treated with either 20 ng·mL⁻¹ stromal cell derived factor 1 (SDF-1, Peprotech) or 10 nM 5-azacytidine (5-aza, Sigma) under normal culture conditions.

### Hybrid Allosteric Receptor (HAR) Construct and Vectors

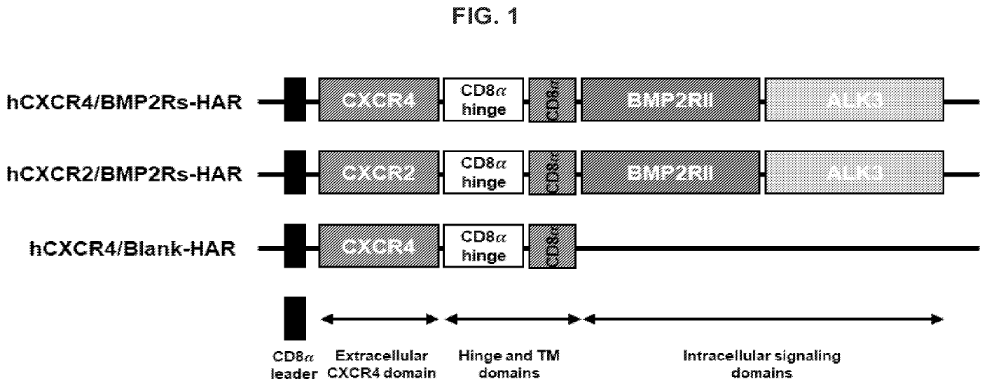

All HAR transgenes were human codon optimized, de novo synthesized, and inserted between KpnI and BamHI in vector pcDNA3.1(+) by GenScript (Piscataway, NJ, USA). As shown in FIG. 1, three HAR constructs were generated. The hCXCR4/BMP2Rs-HAR construct generates the HAR fusion protein containing the extracellular CXCR4, CD8α hinge, CD8α transmembrane, BMP2RII, and ALK3 domains driven by a CMV prornotor. The hCXCR2/BMP2Rs-HAR construct generates the HAR fusion protein containing the extracellular CXCR2, CD8α hinge, CD8α transmembrane, BMP2RII, and ALK3 domains driven by a CMV promotor. The hCXCR4/Blank-HAR construct generates the HAR fusion protein containing the extracellular CXCR4, CD8α hinge, CD8α transmembrane, BMP2RII, and ALK3 domains by CMV promotor. Empty-vector (pcDNA3.1(+)) and full length hCXCR4 (pcDNA3.1(+))-based) were purchased from Addgene. HAR construct sequences and subdomain sequences are shown in Table 1.

TABLE 1
HAR Constructs and Subdomain Sequences.

| Construct | Nucleotide Sequence | Polypeptide Sequence |
|---|---|---|
| hCXCR4/BMP2Rs-HAR | SEQ ID NO: 1 | SEQ ID NO: 2 |
| hCXCR2/BMP2RS-HAR | SEQ ID NO: 3 | SEQ ID NO: 4 |
| hCXCR4/Blank-HAR | SEQ ID NO: 5 | SEQ ID NO: 6 |
| CD8α leader | SEQ ID NO: 7 | SEQ ID NO: 8 |
| hCXCR4 | SEQ ID NO: 9 | SEQ ID NO: 10 |
| hCXCR2 | SEQ ID NO: 11 | SEQ ID NO: 12 |
| CD8α hinge region | SEQ ID NO: 13 | SEQ ID NO: 14 |
| CD8α transmembrane region | SEQ ID NO: 15 | SEQ ID NO: 16 |
| BMP2RII | SEQ ID NO: 17 | SEQ ID NO: 18 |
| Polyglycine Linker | SEQ ID NO: 19 | SEQ ID NO: 20 |
| ALK3 | SEQ ID NO: 21 | SEQ ID NO: 22 |

### Preparation of DIpH Complex

A binary complex of plasmid DNA (pDNA) and lithocholic acid (LCA)-polyethylenimine (PEI) (Ip, provided from Dr. Yeo lab of Purdue University) was first prepared by mixing the two in HEPES buffered saline (HEPES 10 mM, pH 7.4) and incubating at room temperature for 30 min. For preparation of a ternary complex of HAR-pDNA, Ip, and hyaluronic acid (HA) (DIpH), the DIp binary complex was added to HA (20 kDa) solution in HEPES buffered saline and incubated at room temperature (R.T.) for 10 min. For transfection optimization with pDNA/Ip/HA complex, the typical pDNA/Ip/HA ratio was Low-Ip (1/0.3/0.015 w/w/w), High-Ip (1/5/0.25 w/w/w), and two High-Ip-modified ratios (1/10/0.5 w/w/w and 1/20/1 w/w/w) unless specified otherwise.

### Luciferase Reporter Assay

hBMMSCs were cultured in six-well plates, at a cell seeding density of 1×10⁴ cells/cm² (low cell seeding density) or 5×10⁴ cells/cm² (high cell seeding density) and cultured at 37° C. with 5% CO₂. Next, 300 μL of transfection medium containing DIpH, pDNA (luciferase-expressing plasmid DNA (pLuc), pGEM®-luc DNA(Promega, Fitchburg, WI))/Ip/HA equivalent to 400 ng or 1000 ng of pLuc was added followed by a 24 h incubation at 37° C. For the luciferase reporter assay, the typical pDNA/Ip/HA ratio was Low-Ip (1/0.3/0.015 w/w/w) or High-Ip (1/5/0.25 w/w/w). To measure luciferase expression, after 48, 72, or 96 h (including 24 h transfection time), cells were rinsed with PBS and lysed with a cell culture lysis buffer, and the luciferase activity of the cell lysate was measured by the Luciferase activity assay kit (Promega) using a SpectraMax M3 microplate reader (Molecular Devices, Sunnyvale, CA). The total protein amount was measured by Pierce™ BCA Protein Assay. The gene transfection efficiency was expressed as the relative luciferase intensity/mg total protein.

Confirmation of BMP2-related Signaling in hCXCR2-HAR-engineered hMSCs via Interleukin-8 (IL-8) Treatment

HAR-transfected cells were maintained in Dulbecco's Modified Eagle's (DMEM, Gibco Invitrogen), supplemented with 10% fetal bovine serum (FBS, Gibco Invitrogen) at 37° C. in a humidified incubator containing 5% CO₂. The media was refreshed every 3 days. The transfected cells were treated with 20 ng·mL⁻¹ IL-8 (PeproTech) in DMEM supplemented with 10% FBS.

In Vitro Gene Transfection on hMSCs with HARs-pDNA/Ip/HA Ternary Complex

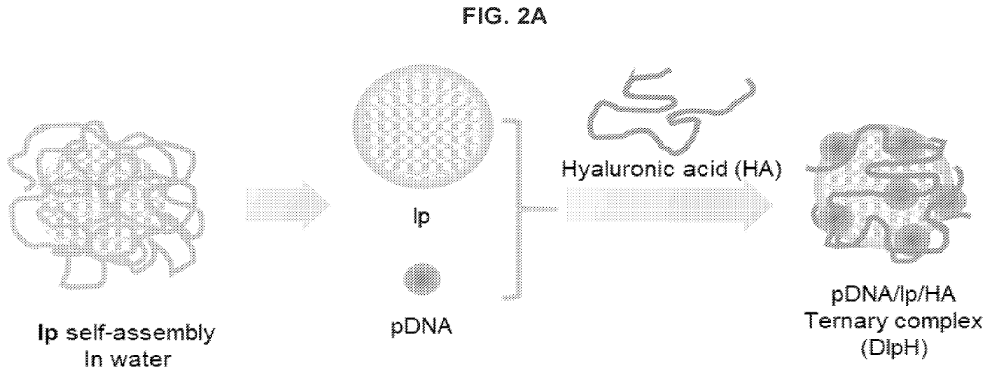

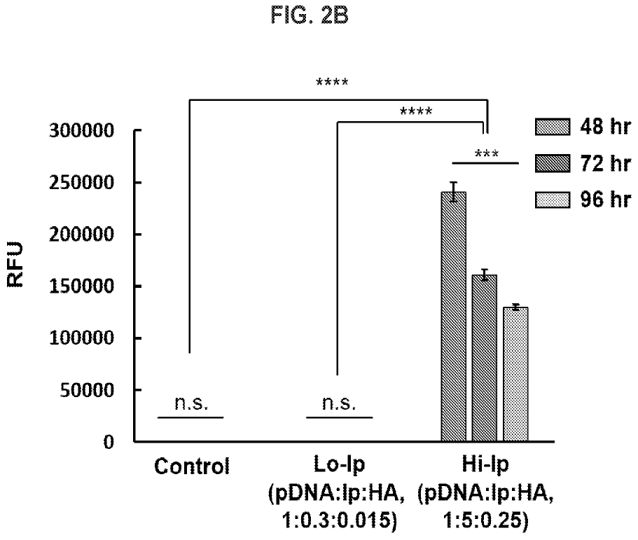

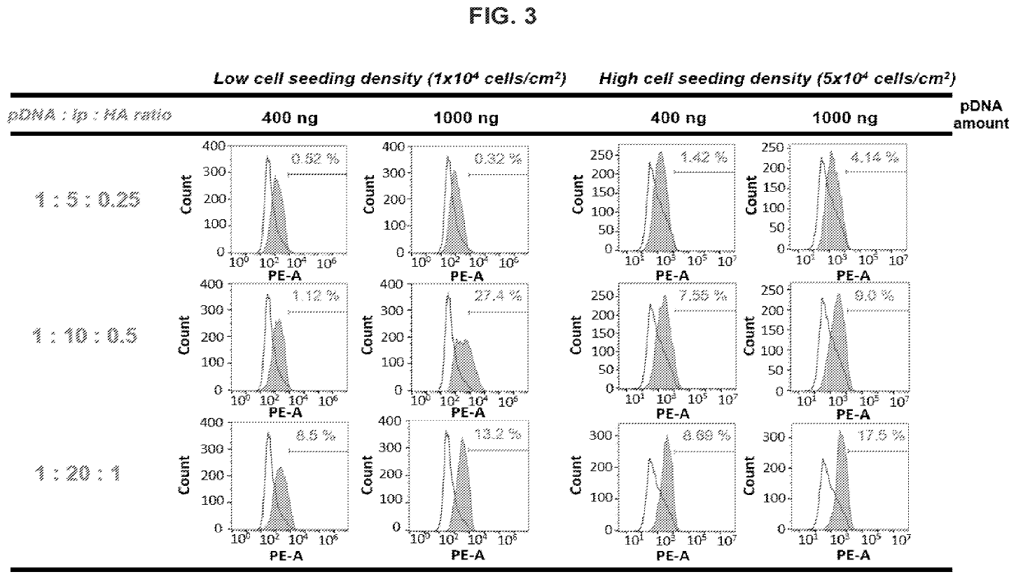

hBMMSCs were seeded in a 24-well plate at both cell seeding density of 1×10⁴ cells/cm² (low cell seeding density) or 5×10⁴ cells/cm² (high cell seeding density) and cultured at 37° C. with 5% CO₂. For MSCs transfection using pDNA/Ip/HA ternary complex, the culture medium was replaced with 300 μL of transfection medium containing DIpH, pDNA/Ip/HA equivalent to 400 ng or 1000 ng of HAR-pDNA, followed by 24 h incubation at 37° C. As shown in FIG. 3, the pDNA/Ip/HA ratio on hBMMSCs was optimized. The transfection medium (DMEM with 10% FBS) was replaced with fresh complete medium, and the cells were allowed to grow for another 48 h (see FIG. 2). The media was refreshed every 3 days.

### Actual Interaction Between HAR-Generated CXCR4 Expressing MSCs and SDF-1 Protein

The HAR transfected MSCs were treated with 20 ng·mL⁻¹ FITC-labeled recombinant human SDF-1 (PeproTech) for 30 min at 37° C. before washing in PBS. Recombinant human SDF-1 was labeled by FluoReporter™ FITC Protein Labeling Kit (Thermo Fisher). The labeling protocol was performed by the manufacturer's instructions. HAR-engineered MSCs with FITC-labeled recombinant human SDF-1 were visualized using fluorescence microscopy (Leica DMI6000) and quantified by BD FACS Cantoll (BD Biosciences).

### Immunocytochemical (ICC) Staining

Samples were fixed with 4% paraformaldehyde, permeabilized with Triton X-100, and blocked with a mixture of bovine serum albumin (BSA). Samples were incubated with primary antibodies (Anti-BMP2R Actin (Abcam, 1:200), Anti-ALK3 antibody (R&D, 1:200), Anti-Smad1/5 (Abcam, 1:200), Anti-cTnT antibody (R&D, 1:200), or Anti-cTnI (Abcam, 1:200)), overnight at 4° C. and washed. Samples were subsequently stained with fluorescently labeled secondary antibodies (Alexa Fluor-488 conjugated goat anti-mouse (Abcam, 1:400), Alexa Fluor-647 conjugated donkey anti-goat (Abcam, 1:400)) for 30 min at RT and 4′,6-diamidino-2-phenylindole dihydrochloride (DAPI) as a nuclear counterstain for 5 min at R.T. Samples were washed and mounted for imaging on a fluorescence microscope (Leica DMI-6000, Leica). The relative surface area of coverage for stains was quantified with the ImageJ software (NIH).

### Immunohistochemical (IHC) Staining

For IHC staining in rat hearts, staining was conducted on a Leica Bond RXm using standard chromogenic methods. For antigen retrieval (HIER), pH 9 EDTA based buffer for 2 h at 70° C. (CD31), or pH 9 EDTA based buffer for 25 min at 94° C. (cTnT), followed by a 30-min antibody incubation (CD31, Abcam, 1:500) or a 20-min antibody incubation (Cardiac Troponin T, Abcam, 1:3000). Antibody binding was detected using an HRP-conjugated secondary polymer (CD31) or an AP-conjugated secondary polymer (cTnT), followed by chromogenic visualization with diaminobenzidine (CD31) or fast red chromogen (cTnT). A Hernatoxylin counterstain was used to visualize nuclei.

RNA Isolation and Quantitative Real-Time Polymerase Chain Reaction (qRT-PCR)

Total RNA was isolated using TRlzol® reagent (Invitrogen) according to the manufacturer's protocol. mRNA was reverse transcribed into complementary DNA (cDNA) using iScript™ cDNA synthesis kit (Bio-Rad), Quantitative PCR analyses were performed using iTaq Universal SYBR Green Supermix (Bio-Rad) with a CFX96 Touch Real-Time PCR Detection System (Bio-Rad) according to the instructions. Target gene expression was normalized to both human and rat glyceraldehyde-3-phosphate dehydrogenase (GAPDH) genes for quantification. Expression levels of Nkx2.5, GATA4, cTnT, and cTnI for human, and vWF, Pecam-1 (CD31), and cTnT for rat, were calculated using 2-ΔΔCT method. The PCR primer sequences are shown in Table 2.

TABLE 2
Human and rat primer sequences for qRT-PCR.
Human Primers

| Gene | Forward primer (5′→3′) | SEQ ID | Reverse primer (5′→3′) | SED ID |
|---|---|---|---|---|
| Nkx2.5 | CACCGGCCAAGTGTGCGTCT | SEQ ID | GCAGCGCGCACAGCTCTTTC | SEQ ID |
| | | NO: 23 | | NO: 4 |
| GATA4 | CGGCGAGGAGGAAGGAGCCA | SEQ ID | TGGGGGCAGAAGACGGAGGG | SEQ ID |
| | | NO: 25 | | NO: 26 |
| cTnT | GGAGGAGAACAGGAGGAAGG | SEQ ID | CTGGATGTAACCCCCAAAATGC | SEQ ID |
| | | NO: 27 | | NO: 28 |
| cTnI | CGTGTGGACAAGGTGGATGA | SEQ ID | GCCGCTTAAACTTGCCTCG | SEQ ID |
| | | NO: 29 | | NO: 30 |
| GAPDH | GGTGAAGCAGGCGTCGGAGG | SEQ ID | GAGGGCAATGCCAGCCCCAG | SEQ ID |
| | | NO: 31 | | NO: 32 |

Rat Primers

| Gene | Forward primer (5′→3′) | SEQ ID | Reverse primer (5′→3′) | SED ID |
|---|---|---|---|---|
| VWF | CCGGAAGCGACCCTCAGA | SEQ ID | CGGTCAATTTTGCCAAAGATCT | SEQ ID |
| | | NO: 33 | | NO: 34 |
| Pecam1 | GGACTGCGCCCATCACTTACC | SEQ ID | AACTTCATCCACCGGGGCTATTAC | SEQ ID |
| | | NO: 35 | | NO: 36 |
| cTnT | GCCAGAGATGCTGAAGATGGT | SEQ ID | GCACCAAGTTGGGCATGAAG | SEQ ID |
| | | NO: 37 | | NO: 38 |
| GAPDH | TCTCTGCTCCTCCCTGTTCTA | SEQ ID | ATGAAGGGGTCGTTGATGGC | SEQ ID |
| | | NO: 39 | | NO: 40 |

### Row Cytometry

Cell surface antigens on cells were evaluated by flow cytometry analysis. For flow cytometry, cells were dissociated by 0.05% trypsin; EDTA (HyClone), washed with PBS, fixed with 4% paraformaldehyde, permeabilized with Triton X-100, and blocked with a mixture of BSA. Next, samples were stained with antibodies against Anti-ALK3 antibody (R&D, 1:200), Anti-Smad1/5 (Abcam, 1:200), Anti-cTnT antibody (R&D, 1:200), and Anti-cTnI (Abeam, 1:200) for 30 min or 1 h at 4° C. Samples were subsequently stained with fluorescently labeled secondary antibodies (Alexa Fluor-488(AF488) conjugated goat anti-mouse (Abcam, 1:400) and Alexa Fluor-647(AF647) conjugated donkey anti-goat (Abcam, 1:400)) for 30 min at 4° C. Corresponding mouse/rabbit isotype antibodies were used as controls. Cell immunotypes were determined by BD FACS Cantoll (BD Biosciences) and the percentage of expressed cell surface antigen was calculated for 10,000 gated-cell events.

### Rat AMI Model and Intramyocardial Transplantation

A well-established and highly reproducible rodent model of MI was used as previously described. Briefly, rats were sedated and placed in an induction chamber delivering isoflurane. The anesthetized rats were intubated using direct laryngoscopy and then transferred to an operating table. The operative procedure began with left thoracotomy in the 4^{th} or 5^{th} intercostal space. After widely incising the pericardium, the left anterior descending artery (LAD) was identified using surgical loupes. The LAD was ligated 1-2 mm from the anterior tip of the left atrial appendage. After confirming successful ligation of the LAD by observation of visible blanching of the LV, and chest was closed in layers. At 30 min after LAD ligation, 1×10⁶ cells of each groups in 100 μL PBS were transplanted by a single intramyocardial injection into the peri-infarct area using 1 mL syringe with a 27-gauge cannula (BD). Four different types of groups were used: (i) PBS; (ii) MSCs only; (iii) hCXCR4-BMP2Rs-HAR-engineered MSCs; or (iv) hCXCR2-BMP2Rs-HAR-engineered MSCs. Each group was followed up for 3 weeks after post-operation. After cell injection, the chest was closed, and the thoracic cavity was evacuated. In addition, some animals received a full thoracotomy with exposure of the heart. The chest was be reopened through the above procedure 3 weeks after single intramyocardial injection for further analysis.

### Echocardiography

The rats were administered inhalant anesthetics (1.5-2% of isoflurane inhalant mixed with 1 L min⁻¹ 100% O₂) to ensure that the anesthetic depth was appropriate. Transthoracic echocardiograms (Visual Sonics Vevo 2100 with a 30-MHz transducer) were performed by an experienced technologist blinded to the study group.

### Histology

Formalin-fixed soft tissue samples were dehydrated through graded alcohols, cleared with xylene, and infiltrated with paraffin wax. The tissue was then embedded in paraffin molds for sectioning on a microtome. Tissue sections, 4-6 μm, were transferred to a water bath and transferred to a glass microscope slide. Slides then had the wax removed, rehydrated, and were stained with Masson's trichrome (MT) protocol. Finally, slides were cover-slipped using permanent, toluene-based mounting media.

### Statistical Analysis

Statistical analyses were performed in GraphPad Prism software (version 8.1.0, GraphPad Software Inc., USA). All data are presented in mean±standard deviation (SD). All data were analyzed with one-way ANOVA with Bonferroni post hoc tests or two-way ANOVA with Bonferroni post hoc tests. Statistical significance threshold of each test was set at P<0.05: ns=not significant, P>0.05; *, P<0.05; **, P<0.01; ***, P<0.001; ****, P<0.0001.

### Example 2

### Structures of Hybrid Allosteric Receptors (HAR) Constructs of BMP2Rs Containing hCXCR4-HAR on Human Mesenchymal Stem Cells (hMSCs)

The HAR construct contained the extra cellular domain of human full-length CXCR4, the CD8α hinge and transmembrane (TM) region, and the tandem BMP2R2 and ALK3 (BMP2R1) intracellular signaling motif (hCXCR4/BMP2Rs-HAR; FIG. 1). For the ALK3 and BMP2R2 co-stimulatory signal to be stimulated by the SDF-1/CXCR4 binding, the anti-CXCR4 extracellular domain portion was fused to the ALK3 and BMP2R2 co-stimulatory domains (hCXCR4/BMP2Rs-HAR). A hCXCR2-HAR containing BMP2R2 and ALK3 signaling motifs (hCXCR2/BMP2Rs-HAR) was constructed as a negative control. This CXCR2 HAR activates the BMP2Rs co-stimulatory domains in responsive to IL-8 instead of SDF-1. CXCR4 without the BMP2Rs internal signaling domain was constructed to subtract the effects of the co-stimulation on the differentiation yield. In addition, the full-length CXCR4 was synthesized as a control. All HAR constructs and the full-length CXCR4 were sub-cloned into the pcDNA™ 3.1 plus mammalian expression vector, where transgene expression was driven off the CMV promoter.

Transfection and Expression of BMP2Rs Containing hCXCR4-HARs in hMSCs

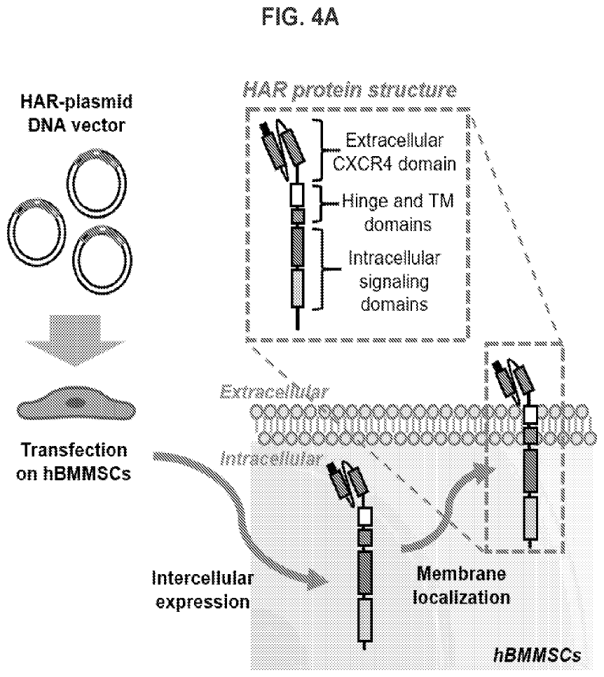

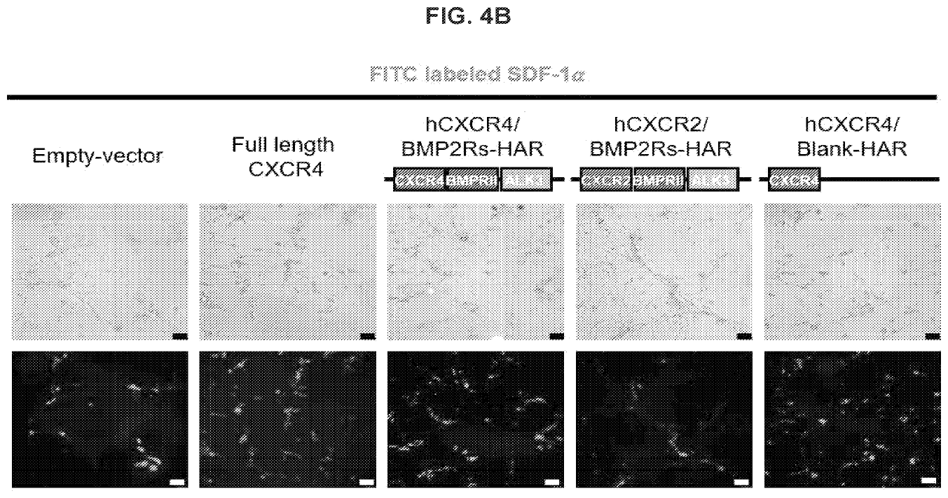

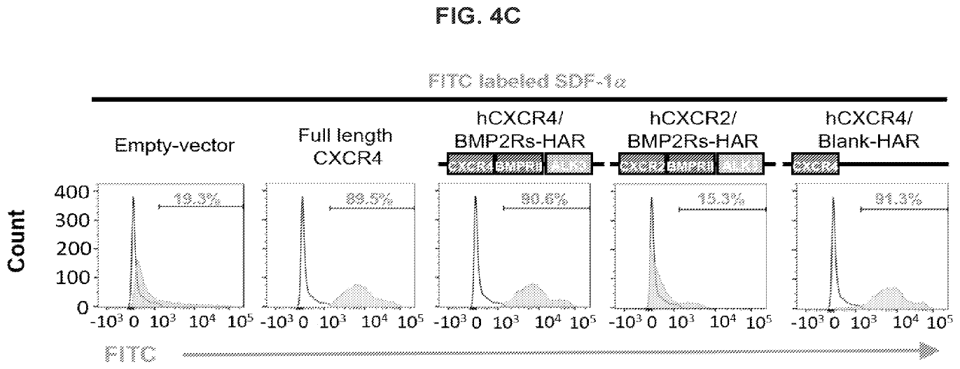

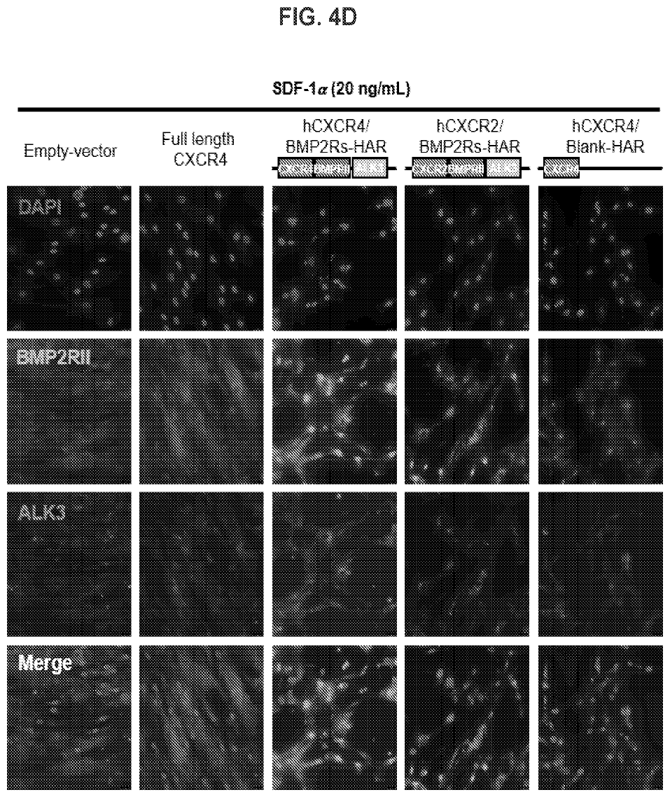

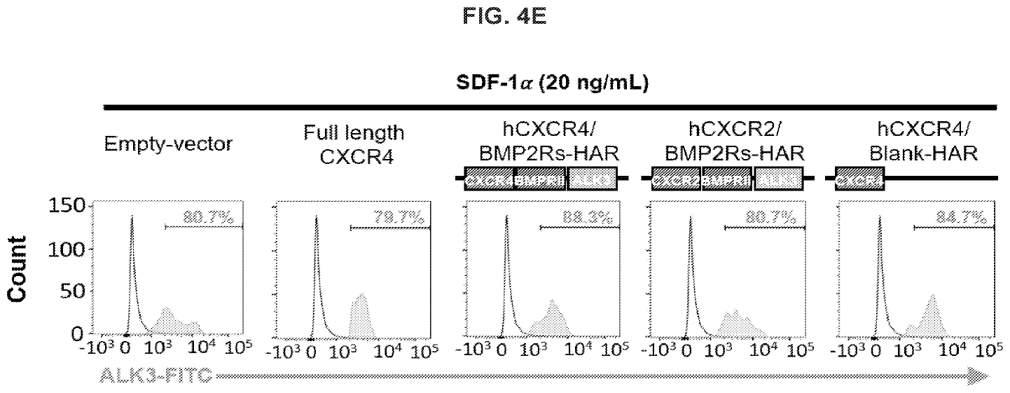

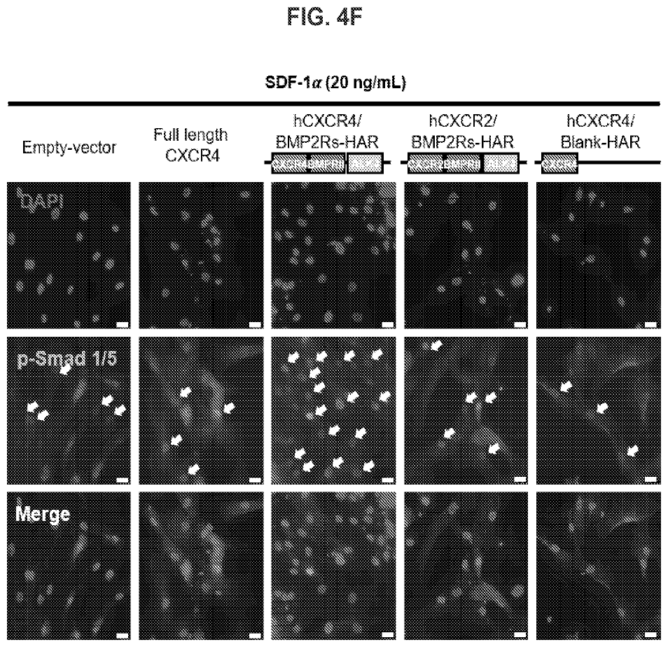

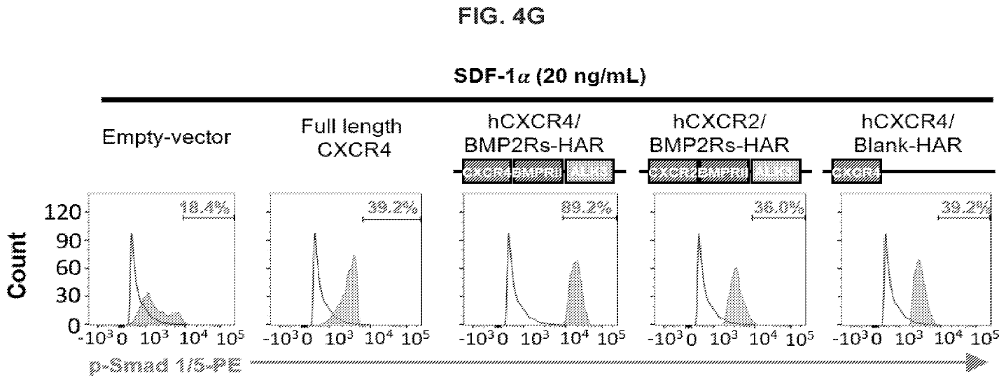

Upon transfection of the hCXCR4/BMP2Rs-HAR encoded plasmid DNA, synthetic HARs proteins were expressed and processed in the hMSCs to locate the CXCR4 portion on the surface of the cells and the so-stimulatory signals inside the cells via CD8 TM (FIG 4A). For better transfection, the transfection conditions were optimized using ternary complexes composed of plasmid DNAs, lithocholic acid (LCA)-conjugated polyethyleneamine (PEI) (Ip), and hyaluronic acid (HA) (generously provided by Dr. Yeo at Purdue University). Transfection was most efficient at the ratio of pDNA:Ip:HA=1:10:0.5 with a cell density of 1×10⁴ cells/cm² (FIGS. 2-3). At the optimized condition, the transfection efficiency of hCXCR4/BMP2Rs-HAR was determined to be 27.4%, significantly greater than other transfection conditions.

Binding of SDF-1 to the HAR-MSCs CXCR4 was examined. First, the MSCs transfected with each of the constructs were incubated with FITC-labeled SDF-1. FITC-SDF-1 was detected on the surface of MSCs in the transfection groups (FIG. 4B). This reveals that fluorescence images of the vector-transfected hMSCs confirmed that hCXCR4-HAR vectors were introduced successfully and expressed continuously (FIG. 4B). To quantify the interaction efficacy by flow cytometry, FITC-conjugated SDF-1 were detected at higher levels in hCXCR4/BMP2Rs-HAR (90.06%), hCXCR4-Blank-HAR (91.3%), and full length CXCR4 (89.5%) than in empty-vector (19.3%) and hCXCR4/BMP2Rs-HAR (15.3%) (FIG. 4C). Importantly, the protein expression of ALK3 and BMP2R2 co-stimulatory domains was confirmed through a phosphorylation of the downstream effector, Smad1/5 induced by these BMP2 signaling in hCXCR4/BMP2Rs-HAR transfected hMSCs with SDF-1 treated culture condition (to mimic CXCR4/SDF-1 interaction). As shown in FIGS. 4D and 4F, the expression of both ALK3 and BMP2R2 protein, even phospho-Smad1/5 (white arrow indicated phospho-Smad1/5 expression in nuclei) were expressed at higher levels in hCXCR4/BMP2Rs-HAR than in other HAR's transfected hMSCs. To quantify the expression of ALK3, one of BMP2R's and phospho-Smad1/5 in groups by flow cytometry, hCXCR4IBMP2Rs-HAR was higher than other HARs transfected hMSCs, including the empty-vector and full length CXCR4 (FIGS. 4E and 4G). Reproducibly, the expression of ALK3 in hCXCR4/BMP2Rs-HAR (88.3%) was slightly higher than with other HAR's transfected hMSCs, as assessed by flow cytometry. Interestingly, unlike ALK3, the expression of phospho-Smad1/5 in hCXCR4/BMP2Rs-HAR (89.2%) was significantly higher than other HAR's transfected hMSCs. These results demonstrate that hCXCR4/BMP2Rs-HAR-mediated signals on hMSCs are functionally well-expressed.

In Vitro Cardiomyocyte Differentiation by hMSCs Expressing hCXCR4/BMP2Rs-HAR

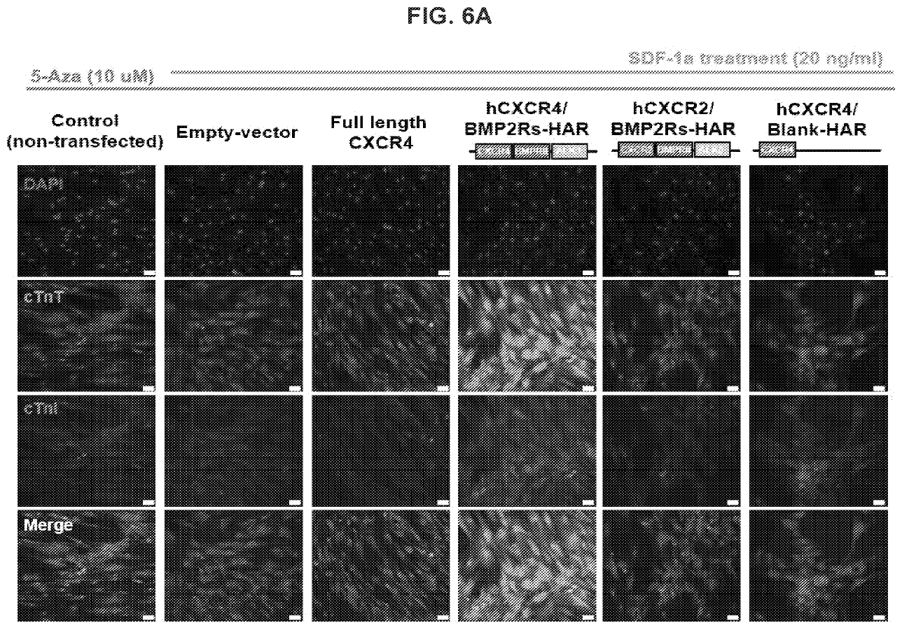

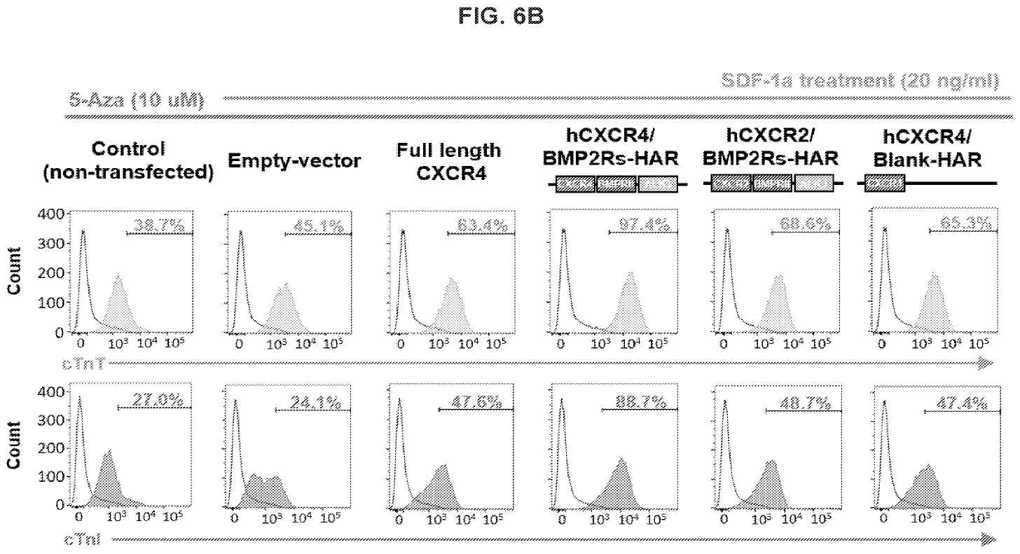

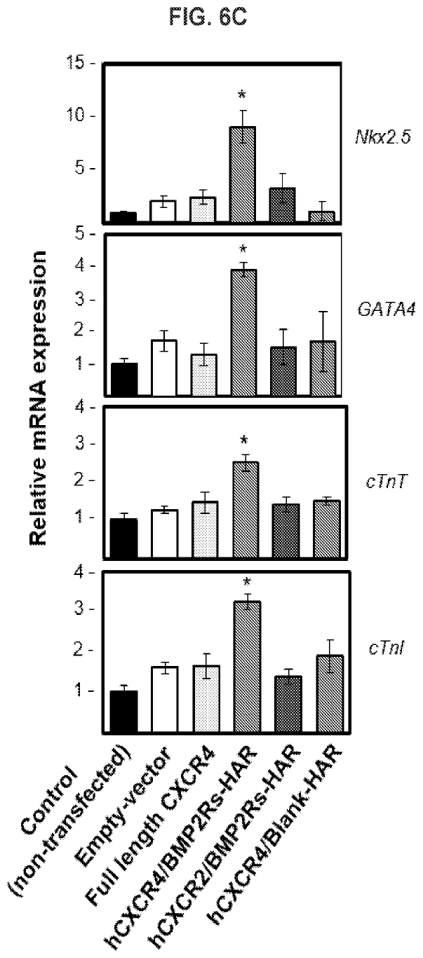

To compare the cardiomyocyte differentiation occurring as a result of transfection with that in all groups including 5-azacytidine (5-aza) treated conventional culture method, the empty vector, full length CXCR4, hCXCR4/BMP2Rs-HAR, hCXCR2/BMP2Rs-HAR, and hCXCR4/Blank-NAR vectors were transfected into hMSCs. After HAR's transfection, the hMSCs in all groups were treated with 10 μM 5-aza for 24 hours. Next, the cells were cultured with or without SDF-1 for 14 days to assess the inductive cardiomyocyte differentiation effect. As shown in FIG. 6A, the protein expression of human cardiac troponin T (cTnT) and cardiac troponin I (cTnI) were expressed at higher levels in hCXCR4/BMP2Rs-HAR than in other transfected hMSCs, even in conventional cultures. To quantify the expression of cTnT and cTnI protein in all groups by flow cytometry, hCXCR4/BMP2Rs-HAR (cTnT; 97.4%, cTnI; 88.7%) was significantly higher than other HAR's transfected hMSCs, including empty-vector and full length CXCR4, as well as conventional cultures (FIG. 6B). Additionally, qRT-PCR analysis showed that cardiomyocyte markers such as Nkx2.5, GATA4, cTnT, and cTnI were expressed at higher levels in hCXCR4/BMP2Rs-HAR-engineered hMSCs (Nkx2.5; 9.07±1.53 fold-change, GATA4; 3.91±0.23 fold-change, cTnT; 2.53±0.21 fold-change, and cTnI; 3.25±0.19 fold-change, vs. control (none-engineered group)) than in the other groups, and the expression of these genes have no significant difference between all other groups, excluding hCXCR4/BMP2Rs-HAR-engineered hMSCs (FIG. 6C). These results demonstrate that hCXCR4/BMP2Rs-HAR-engineered hMSCs enhance cardiomyocyte differentiation more significantly than other HAR's transfected hMSCs, including empty-vector and full length CXCR4.

In Vivo Cardiac Functional Improvement by hMSCs Expressing hCXCR4-BMP2Rs-HAR

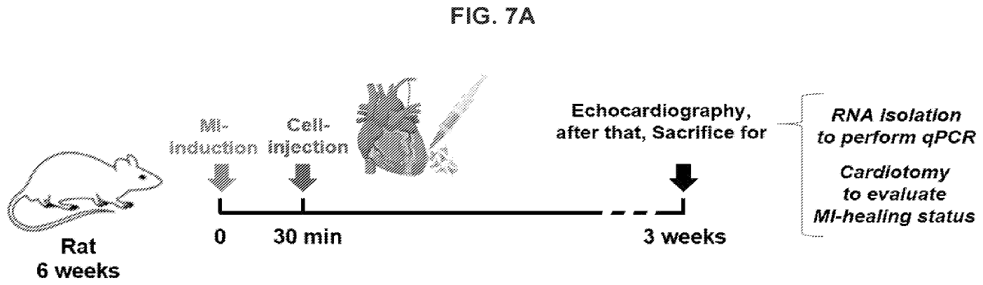

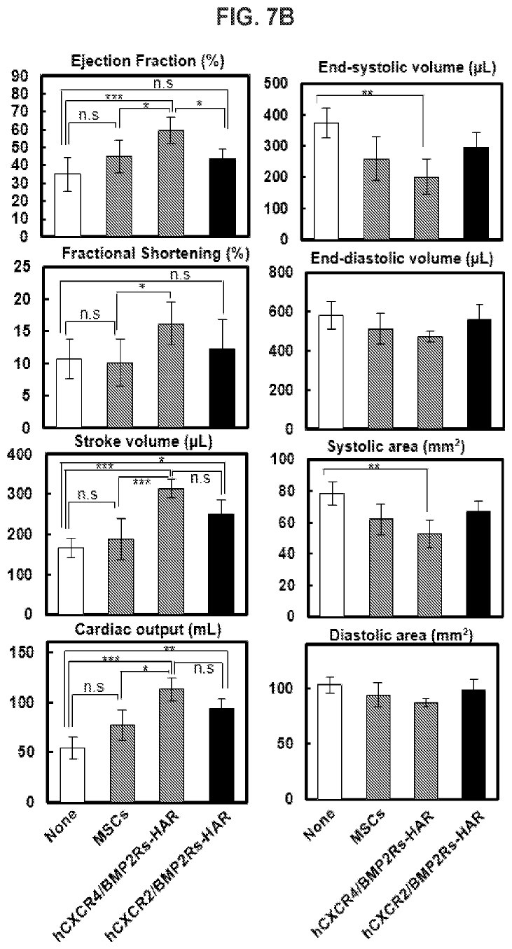

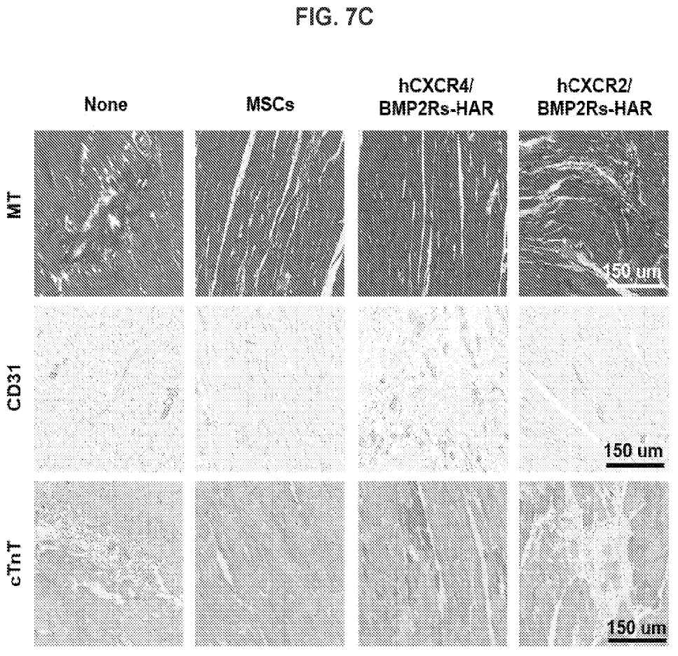

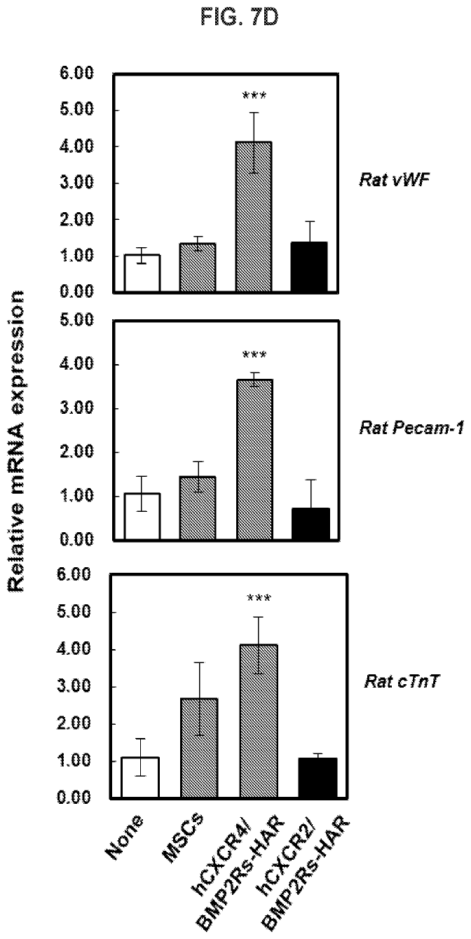

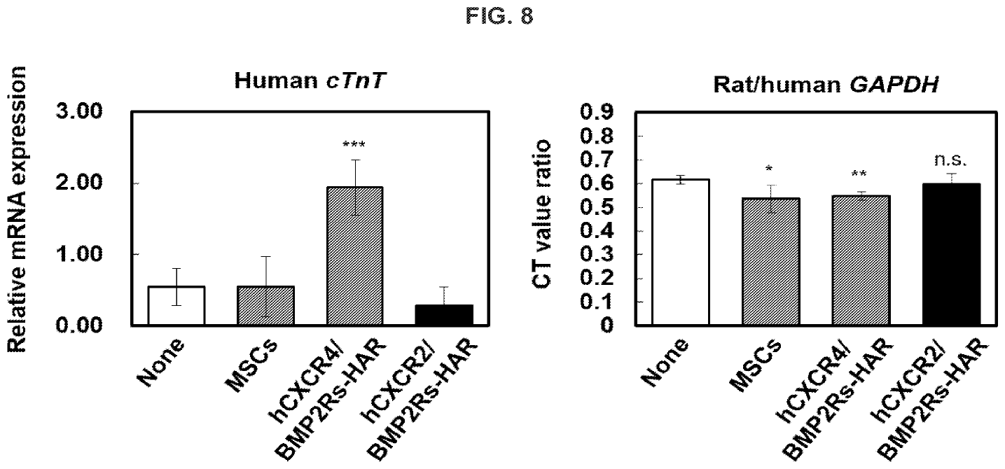

To verify in vivo cardiac functional improvement via an enhanced cardiomyocyte differentiation by hCXCR4/BMP2Rs-HAR-engineered hMSCs, hMSCs, hCXCR4/BMP2Rs-HAR engineered hMSCs, and hCXCR2/BMP2Rs-NAR-engineered hMSCs were directly transplanted in the rat hearts after 30 min of LAD coronary artery ligation (FIG. 7A). As shown in FIG. 7B, the echocardiography studies showed that a damaged systolic function characterized by a significant improvement of the ejection fraction was observed in the transplantation of hCXCR4/BMP2Rs-HAR-engineered hMSCs (59.72±7.26%), compared to MI-induced rat heart (34.88±9.34%). However, the ejection fraction of each hMSCs only (44.66±9.12%) and hCXCR2/BMP2Rs-HAR-engineered hMSCs (43.42±5.86%)-injected groups were similar with MI-induced rat heart. Other parameters of the in vivo cardiac functional improvement, including fractional shortening, stroke volume, and cardiac output, were significantly improved compared to other groups in hCXCR4/BMP2Rs-HAR-engineered hMSCs. Moreover, there results of the echocardiography demonstrated that hCXCR2/BMP2Rs-HAR-engineered hMSCs can effectively reduce LV external end-systolic volume/dimension, compared with MI-induced group. But hCXCR2/BMP2Rs-HAR-engineered hMSCs can significantly reduce LV external end-systolic volume/dimension and LV external end-diastolic volume/dimension, compared with MI-induced group. However, all parameter in each MSCs only and hCXCR2/BMP2Rs-HAR-engineered hMSCs injected groups were similar with the MI-induced group. These results suggest that hCXCR2/BMP2Rs-HAR induced cardiomyocyte differentiation from hMSCs can attenuate cardiac dilation. After the observation of echocardiography from all groups, the hearts of groups were excised and tissues were processed for Masson's trichrome, CD31, and cTnT staining, and utilized to isolate RNA for performing rat angiogenesis (vWF and Pecam-1) and cardiomyocyte (cTnT) markers by qPCR (FIGS. 7C and 7D).

The infarcted site in the left ventricle was examined by Masson's trichrome staining. The representative images of MT show that infarcted site was significantly deceased in MSCs only- and hCXCR2/BMP2Rs-HAR-engineered hMSCs-injected groups, compared with the others. Rat anti-CD31 staining revealed that vessel density as indirect parameter to proof the healing was significantly increased in hCXCR2/BMP2Rs-HAR-engineered hMSCs groups, compared with the others. Additionally, rat cTnT, cardiomyocyte marker, was significantly increased in hCXCR2/BMP2Rs-HAR-engineered hMSCs groups, compared with the others. To confirm the effects of hCXCR2/BMP2Rs-HAR-engineered hMSCs on inducing the rat cardiac healing, the mRNA levels of rat angiogenesis (vWF and Pecam-1) and cardiomyocyte (cTnT) markers were examined by qPCR (FIG. 7D). The results showed that hCXCR4/BMP2Rs-HAR-transfected hMSCs injected group was significantly or trendily increased compared to the other groups. Accordingly, these findings demonstrate that hCXCR4/BMP2Rs-HAR-engineered hMSCs improve the cardiac functional recovery via the improvement of rat angiogenesis and cardiomyocyte differentiation.

Taken together, these results suggest that the healing effect of hCXCR4/BMP2Rs-HAR-engineered MSCs on AMI model is mediated by the induction of cardiac differentiation from human mesenchymal stem cells via a mechanism involving the BMP2R's and Smad 1/5-mediated signaling pathway through endogenous SDF-1 to exogenous engineered CXCR4 binding.

### Example 3

Chimeric antigen receptor (CAR) is an innovative technology that enables cells to recognize and kill specifically to its targeted tumors. CAR technologies are easy and effective way to provide control over the cell fate and function of the modified cells. As is well known that CAR technology is used limitedly for anti-tumor treatment using T cells, previously mentioned as “CAR-T cell therapy.” Although CAR-T cell therapy has proved successful in treating certain tumors, but these CAR technologies have very limited accession in stem cell therapies for regenerative medicine. As an exception, human pluripotent stem cells (hPSCs)-derived immune cells with CAR technologies is just being investigated for the utilization of anti-tumor treatment. So far, no research has direct engineering of stem cells through CAR technologies for the potential of stem cell therapy in regenerative medicine, even though hMSCs, one of therapeutic stem cells are the most frequently applied cells in cell-based regenerative medicine. In addition, to date, over 900 clinical trials worldwide have utilized hMSCs to treat aging-associated diseases and other various diseases, such as cartilage repair, diabetes, cardiovascular diseases, and immune-related disorders. Therefore, through the clinical promise of hMSCs and the advantage of CAR technology, the enormous effort will be applied to develop stem cell-therapies via that of CAR technology for enhancing the targeted specific cell differentiation ability and restoring tissue's own function.

HARs containing both extracellular CXCR4 and intracellular BMP2 receptors signaling moieties are potent molecules able to increase cardiomyocyte differentiation in vitro from hMSCs and restoring in vivo cardiac function after induction of AMI by HAR-based stem cell therapy. Human bone marrow-derived mesenchymal stem cells (hBM-MSCs) expressing hCXCR4/BMP2Rs-HAR, outfitted with extracellular hCXCR4 and intracellular BMP2Rs (ALK3, BMP2RI, and BMP2RII) costimulatory signaling domain in tandem, and enhanced cardiomyocyte differentiation in vitro and demonstrated the improved cardiac function in vivo in a rat AMI model.

Many preclinical studies reported that MSCs have the beneficial effects on left ventricular (LV) remodeling and the recovery of cardiac performance following MI, resulting by paracrine stimulation. However, to obtain a large quantity of therapeutic hMSCs, several weeks of culture expansion of cells are necessary prior to administration or transplantation. Because of these long-term of culture expansion, unfortunately, MSCs have downregulated the expression of CXCR4 on cell surface. As previously mentioned, SDF-1/CXCR4 interaction plays an important role in MSCs homing/localization and tissue healing on AMI sites. Therefore, firstly, it was determined whether actual SDF-1/CXCR4 binding can be enhanced by the engineered extracellular hCXCR4 in hCXCR4/BMP2Rs-HAR engineered MSCs, through SDF-1 protein. Consistent with previous reports, additional hCXCR4 generated from hCXCR4/BMP2Rs-HAR can affect the interaction with FITC-labeling SDF-1 protein in in vitro culture conditions. As a result, a significant increase in BMP2Rs, such as ALK3 (BMP2RI) and BMP2RII expression during cultivation was observed with SDF-1 treatment, after that the downstream effector of BMP2 signal, phospho-Smad1/5 was increased in hCXCR4/BMP2Rs-HAR. Although the differences in BMP2Rs expression within their respective groups have been shown as a narrow range, but hCXCR4/BMP2Rs-HAR expressing MSCs had large differences in actual expression of phospho-Smad1/5 between groups. Additionally, many studies demonstrated that various transfection/transduction methods of CXCR4 have been shown to increase CXCR4 expression in MSCs. However, there are no reports of utilizing CAR approaches to express extracellular resign of CXCR4 in MSCs or any other types of cells. Therefore, HAR-engineered hMSCs for expressing hCXCR4 and BMP2Rs were created.

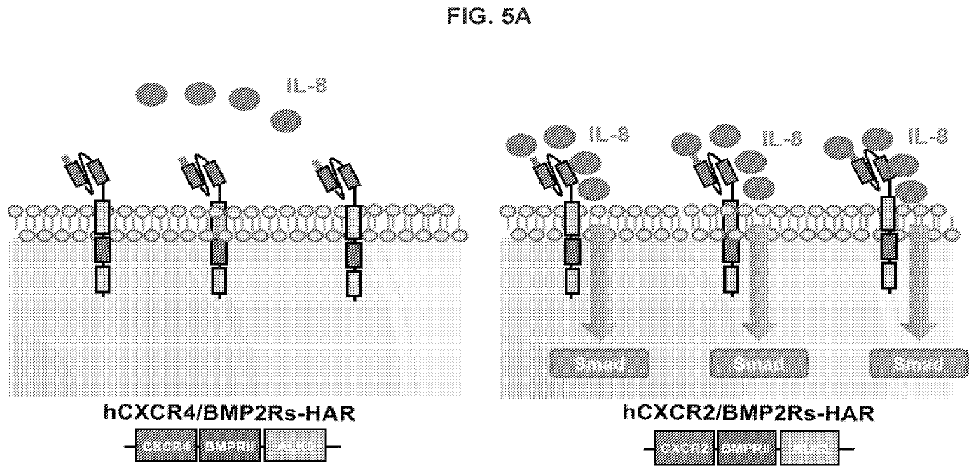

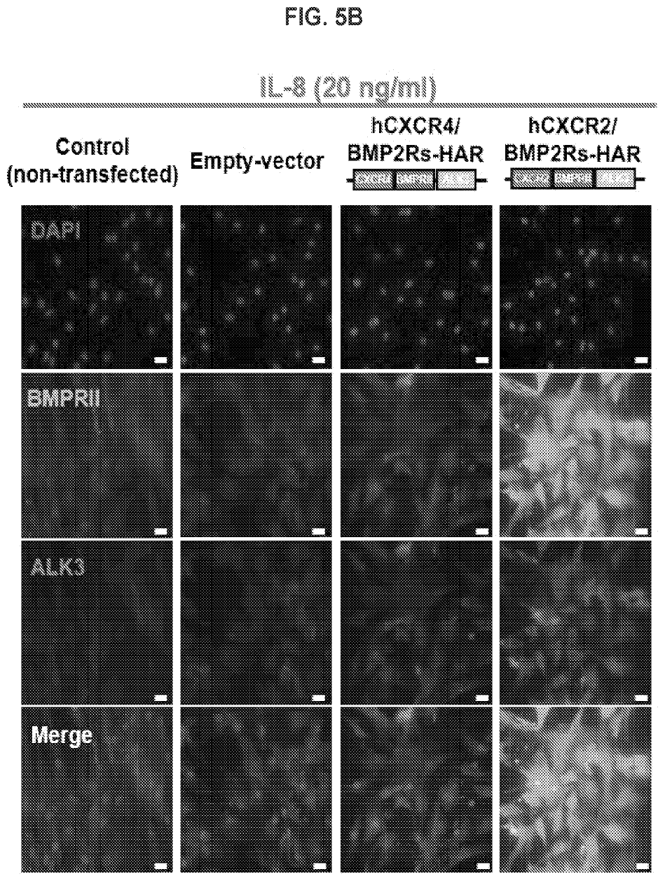

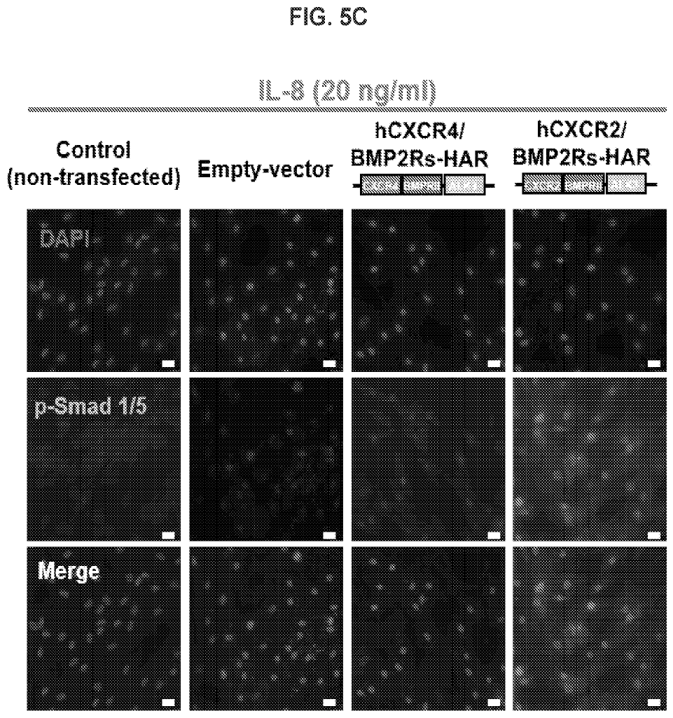

Most researchers have generally focused on developing methods for cardiomyocyte differentiation from hMSCs through the induction of chemicals, cytokine, and simulated cardiac microenvironment, Based on numerous studies, the most typical approach utilizes 5-aza, DNA demethylating agent to induce differentiation of MSCs into cardiomyocytes via direct expression induction of cardiac-specific genes. However, despite the progress being achieved, a commonly accepted standard of clinical-grade cells still remains obscure in the utilization of hMSCs, due to heterogeneity during the period of cardiomyocyte differentiation. Over the past decades, genetic engineering-based techniques have become a novel strategy which can induce cardiomyocytes and improve the cardiac regeneration via gene-transduced MSCs. Among several cardiomyogenic induced genes, BMP2 signaling with BMP2 type I (ALK3) and II receptor inserting in hCXCR4-mediated HAR for cardiomyocyte differentiation from MSCs were utilized. In terms of cardiac development and cardiomyocyte differentiation from mesodermal lineage cells, well-identified BMPs signal play a pivotal role both in self-renewal of stem cells and their differentiation into cardiomyocytes. MSCs by inducing BMP2Rs to activate cardiac gene networks by hCXCR4-mediated HAR could obtain cardiomyocyte differentiation, through the downstream effector, Smad1/5 induced by these BMP2 signaling in hCXCR4/BMP2Rs-HAR. Another hCXCR series-mediated HAR, which is human IL-8 antigen receptor, hCXCR2, hCXCR2-mediated HAR-engineered MSCs had no effect on BMP2 signaling with BMP2 type I (ALK3) and II in the absence of IL-8. Therefore, the status of cardiomyocyte differentiation in hCXCR2/BMP2Rs-HAR-engineered MSCs was similar to 5-aza-treated non-transfected MSCs, due to no stimulation with IL-8. Oppositely, when hCXCR2/BMP2Rs-HAR-engineered MSCs was by IL-8, the high level of BMP2Rs expression with phospho-Smad1/5 in hCXCR2/BMP2Rs-HAR-engineered MSCs was observed (FIG. 5).

hCXCR4/BMP2Rs-HAR-engineered hMSCs show in vivo functionally cardiac improvement using rat AMI model with cardiac function-related various parameters. The possible reason for cross-species transplantation using HAR-engineered human cell and rat animal model is that both SDF-1 and CXCR4 show a high degree of sequence homology in humans and rats. Therefore, this result suggests that these HAR-engineered MSCs transplantation might be more effective to in vivo stimuli enhancing cardiac functional recovery by cardiomyogenic differentiation, than conventional MSCs transplantation. Another CXCR series-HAR, hCXCR2/BMP2Rs-HAR-engineered hMSCs-injected group show partially in vivo cardiac improvement compared to MSCs and hCXCR4/BMP2Rs-HAR-engineered MSCs, even though the improvement of in vitro cardiomyocyte differentiation by hCXCR2/BMP2Rs-HAR-engineered hMSCs could not be confirmed. To support the in vivo partial therapeutic effect of CXCR2-engineered MSCs, there is some evidence that endothelial cells overexpressing IL-8 receptor, CXCR2 reduce post MI-induced cardiac dysfunction with cardioprotective effects. In addition, Xu et al. reported that overexpression of CXCR1/CXCR2 on MSCs may be an effective treatment for AMI. These studies demonstrate that fusion receptors, HAR containing both human CXCR4 and BMP2 receptors signaling moieties, are potent molecules able to increase cardiomyocyte differentiation in vitro from hMSCs and enhance functionally cardiac improvement in vivo against AMI and other cardiovascular diseases or disorders.

### Example 4

Alternative hybrid allosteric receptor (HAR) constructs can be designed by combining various extracellular, intracellular, transmembrane, leader sequences, and linkers. In one embodiment a HAR construct can be created as shown in the following schematic:

Various extracellular, intracellular, transmembrane, leader sequences, and linkers useful for creating HAR constructs are shown in Table 3.

TABLE 3
Extracellular, Intracellular, Transmembrane, Leader Sequences, and Linkers for
Creating HAR Constructs

| | | |
|---|---|---|
| Domain | Abbreviation | Accession Number(s) |

Extracellular and/or Intracellular Domains

| | | |
|---|---|---|
| Chemokine (C-C motif) receptor 1 | (CCR1) | NP_001286.1 |
| Chemokine (C-C motif) receptor 2 | (CCR2) | NP_001116513.2 |
| Chemokine (C-C motif) receptor 3 | (CCR3) | NP_001158152.1 |
| Chemokine (C-C motif) receptor 4 | (CCR4) | NP_005499.1 |
| Chemokine (C-C motif) receptor 5 | (CCR5) | NP_000570.1 |
| Chemokine (C-C motif) receptor 6 | (CCR6) | NP_004358.2 |
| Chemokine (C-C motif) receptor 7 | (CCR7) | NP_001288643.1 |
| Chemokine (C-C motif) receptor 8 | (CCR8) | NP_005192.1 |
| Chemokine (C-C motif) receptor 9 | (CCR9) | NP_001243298.1 |
| Chemokine (C-C motif) receptor 10 | (CCR10) | NP_057686.2 |
| Chemokine (C-X-C motif) receptor 1 | (CXCR1) | NP_000625.1 |
| Chemokine (C-X-C motif) receptor 2 | (CXCR2) | NP_001161770.1 |
| Chemokine (C-X-C motif) receptor 3 | (CXCR3) | NP_001136269.1 |
| Chemokine (C-X-C motif) receptor 4 | (CXCR4) | NP_001008540.1 |
| Chemokine (C-X-C motif) receptor 5 | (CXCR5) | NP_001707.1 |
| Chemokine (C-X-C motif) receptor 6 | (CXCR6) | NP_001373364.1 |
| C-X3-C motif chemokine receptor 1 | (CX3CR1) | NP_001164642.1 |
| Chemokine (C motif) XC receptor 1 | (XCR1) | NP_001019815.1 |
| Atypical chemokine receptor 1 | (ACKR1) | NP_001116423.1 |
| Atypical chemokine receptor 2 | (ACKR2) | NP_001287.2 |
| Atypical chemokine receptor 3 | (ACKR3) | NP_064707.1 |
| Atypical chemokine receptor 4 | (ACKR4) | NP_057641.1 |
| C-C Motif Chemokine Receptor Like 2 | (CCRL2) | NP_001124382.1 |
| Activin A Receptor Type 1 | (ACVR1) | NP_001096.1 |
| Bone morphogenetic protein receptor type 1A | (BMPRIA (ALK3)) | NP_033888.2 |
| Bone morphogenetic protein receptor type 1B | (BMPRIB (ALK6)) | NP_001264145.1 |
| Bone morphogenetic protein receptor type 2 | (BMPR2) | NP_001195.2 |
| Activin A receptor type 2A | (ACVR2A) | NP_001265508.1 |
| Activin A receptor type 2B | (ACVR2B) | NP_001097.2 |
| Transforming growth factor beta receptor 1 | (TGFBR1) | NP_001124388.1 |
| Transforming growth factor beta receptor 2 | (TGFBR2) | NP_001020018.1 |
| Vascular endothelial growth factor receptor 1 | (VEGFR1) | NP_001153392.1 |
| Vascular endothelial growth factor receptor 2 | (VEGFR2) | NP_002244.1 |
| Vascular endothelial growth factor receptor 3 | (VEGFR3) | XP 016864757.1 |
| Epidermal growth factor receptor | (EGFR) | NP_001333826.1 |
| Erb-B2 receptor tyrosine kinase 2 | (ErbB2) | NP_001005862.1 |
| Erb-B2 receptor tyrosine kinase 3 | (ErbB3) | NP_001005915.1 |
| Erb-B2 receptor tyrosine kinase 4 | (ErbB4) | NP_001036064.1 |
| Platelet-derived growth factor receptor A | (PDGFRα) | NP_001334756.1 |
| Platelet-derived growth factor receptor B | (PDGFRβ) | NP_001334756.1 |
| Fibroblast growth factor receptor 1 | (FGFR1) | NP_001167534.1 |
| Fibroblast growth factor receptor 2 | (FGFR2) | NP_000132.3 |
| Fibroblast growth factor receptor 3 | (FGFR3) | NP_000133.1 |
| Fibroblast growth factor receptor 4 | (FGFR4) | NP_001278909.1 |
| Hepatocyte growth factor | (HGF) | NP_000592.3 |
| Tropomyosin-related kinase A | (TrkA) | NP_001007793.1 |
| Tropomyosin-related kinase B | (TrkB) | NP_001007098.1 |
| Tropomyosin-related kinase C | (TrkC) | NP_001007157.1 |
| p75 neurotrophin receptor | (p75NTR) | NP_150086.2 |
| Erythropoietin receptor | (EPOR) | NP_000112.1 |
| Growth hormone receptor | (GHR) | NP_000154.1 |
| Prolactin receptor | (PRLR) | NP_000940.1 |
| Thrombopoietin receptor | (TOPR) | NP_005364.1 |
| Granulocyte colony-stimulating factor receptor | (GCSFR) | NP_000751.1 |
| Leukemia inhibitory factor receptor | (LIFR) | NP_001121143.1 |
| Ciliary neurotrophic factor receptor | (CNTER) | NP_001193940.1 |
| Cardiotrophin-like cytokine factor 1 | (CLCF1) | NP_001159684.1 |
| Oncostatin M receptor | (OSMR) | NP_001161827.1 |
| Interferon alpha receptor 1 | (IFNAR1) | NP_000620.2 |
| Interferon alpha receptor 2 | (IFNAR2) | NP_000865.2 |
| Glycoprotein 130 | (gp130) | NP_001177910.1 |
| Glycoprotein 140 | (gp140) | NP_073753.3 |
| Integrin subunit alpha 1 | (ITGA1) | NP_852478.1 |
| Integrin subunit alpha 2 | (ITGA2) | NP_002194.2 |
| Integrin subunit alpha 3 | (ITGA3) | NP_002195.1 |
| Integrin subunit alpha 4 | (ITGA4) | NP_000876.3 |
| Integrin subunit alpha 5 | (ITGA5) | NP_002196.4 |
| Integrin subunit alpha 6 | (ITGA6) | NP_000201.2 |
| Integrin subunit alpha 7 | (ITGA7) | NP_001138468.1 |
| Integrin subunit alpha L | (ITGAL) | NP_001107852.1 |
| Integrin subunit alpha M | (ITGAM) | NP_000623.2 |
| Integrin subunit alpha IIB | (ITGAIIB) | NP_000410.2 |
| Integrin subunit alpha V | (ITGAV) | NP_001138471.2 |
| Integrin subunit beta 1 | (ITGB1) | NP_002202.2 |
| Integrin subunit beta 2 | (ITGB2) | NP_000202.3 |
| Integrin subunit beta 3 | (ITGB3) | NP_000203.2 |
| Integrin subunit beta 4 | (ITGB4) | NP_000204.3 |
| Integrin subunit beta 5 | (ITGB5) | NP_001341693.1 |
| Integrin subunit beta 6 | (ITGB6) | NP_000879.2 |
| Integrin subunit beta 8 | (ITGB8) | NP_002205.1 |
| Interleukin 1 receptor type 1 | (IL1R1) | NP_000868.1 |
| Interleukin 1 receptor type 2 | (IL1R2) | NP_001248348.1 |
| Interleukin 2 receptor alpha | (IL2RA) | NP_000408.1 |
| Interleukin 2 receptor beta | (IL2RB) | NP_000869.1 |
| Interleukin 2 receptor gamma | (IL2RG) | NP_000197.1 |
| Interleukin 3 receptor alpha | (IL3RA) | NP_001254642.1 |
| Interleukin 4 receptor | (IL4R) | NP_000409.1 |
| Interleukin 5 receptor alpha | (IL5RA) | NP_000555.2 |
| Interleukin 6 receptor | (IL6R) | NP_000556.1 |
| Interleukin 7 receptor | (IL7R) | NP_002176.2 |
| Interleukin 9 receptor | (IL9R) | NP_002177.2 |
| Interleukin 11 receptor alpha | (IL11RA) | NP_001136256.1 |
| Interleukin 12 receptor beta 1 | (IL12RB1) | NP_001276952.1 |
| Interleukin 13 receptor alpha 1 | (IL13RA1) | NP_001551.1 |
| Interleukin 13 receptor alpha 2 | (IL13RA2) | NP_000631.1 |
| Interleukin 15 receptor | (IL15R) | NP_001258426.1 |
| Interleukin 18 receptor 1 | (IL18R1) | NP_001269328.1 |
| Interleukin 21 receptor | (IL21R) | NP_068570.1 |
| Interleukin 23 receptor | (IL23R) | NP_653302.2 |
| Interleukin 27 receptor alpha | (IL27RA) | NP_004834.1 |
| Interleukin 10 receptor alpha | (IL10RA) | NP_001549.2 |
| Interleukin 10 receptor beta | (IL10RB) | NP_000619.3 |
| Interleukin 20 receptor alpha | (IL20RA) | NP_001265651.2 |
| Interleukin 20 receptor beta | (IL20RB) | NP_653318.2 |
| Interleukin 22 receptor alpha 1 | (IL22RA1) | NP_067081.2 |
| Interleukin 22 receptor alpha 2 | (IL22RA2) | NP_443194.1 |
| Interleukin 28 receptor alpha | (IL28RA) | NP_734464.1 |
| Interleukin 28 receptor beta | (IL28RB) | NP_001333866.1 |

Leader Signal Domains
MALPVTALLLPLALLLHAARP
MACLGFLLPVGFLLLISTVAG
MGVLLTQRTLLSLVLALLFPSMASM
MKCLLYLAFLFIGVNC
METDTLLLWWVLLLWVPGSTGD
MGWSCIILFLVATATGVHS
MRAWIFFLLCLAGRALA
MWWWRLWWLLLLLLLLWPMVWA
MDMRVPAQLLGLLLLWLRGARC
MPLLLLLPLLWAGALA
MDAMKRGLCCVLLLCGAVFVSPS
MAFLWLLSCWALLGTTFG
MNLLLILTFVAAAVA
MYRMQLLSCIALSLALVTNS
MGVKVLFALICIAVAEA
MKWVTFISLLFSSAYS
MKTIIALSYIFCLVLG
MALWMRLLPLLALLALWGPDPAAA
MKPIFLVLLVVTSAYA
Transmembrane Domains

| | |
|---|---|
| CD8α | NP_001139345.1 |
| CD28 | NP_001230006.1 |
| CD3ζ | NP_000725.1 |
| ICOS | NP_036224.1 |
| DAP-12-KIR2DS2 multimeric transmembrane | NP_001166985.1/ |
| | NP_001278624.1 |
| FcϵR1 α chain | NP_034314.2 |
| FcϵR1 β chain | NP_000130.1 |
| FcϵR1 γ chain | NP_034315.1 |

Spacers/Linkers

| | |
|---|---|
| GGGSGGGSGGGS | |
| HHHHHHIIKIIK | Gag Spacer Peptide P1 |
| IgG1 Fc region | |
| IgG2 Fc region | |
| IgG3 Fc region | |
| IgG4 Fc region | |
| IgE Fc region | |
| IgM Fc region | |
| IgA Fc region | |
| Modified IgG1 Fc region | |
| Modified IgG2 Fc region | |
| Modified lgG3 Fc region | |
| Modified lgG4 Fc region | |
| Modified IgE Fc region | |
| Modified IgM Fc region | |
| Modified IgA Fc region | |

### Example 5

### Development of Allogeneic HAR-MSC CXCR4-BMP2RII/ALK3

A HAR construct has been developed and tested that facilitates the differentiation of MSCs in response to SDF-1. This construct provides the basis for a new viral gene construct. An adeno-associated virus (AAV) vector will be used to develop allogeneic umbilical cord-derived MSCs (UC-MSCs). The characteristics and differentiation potential of HAR-UCMSCs will be compared with HAR-BMMSCs.

### Umbilical Cord Mesenchymal Stem Cell Culture and Maintenance

Human UCMSCs will be purchased from the American Type Cell Culture (ATCC). Umbilical cord (UC) tissue is considered an attractive source for MSCs for use in allogeneic stem cell therapy. MSCs from UCs have several advantages compared to other sources: (1) unlimited availability, as these cells are isolated from a source otherwise discarded; (2) UC-stem cells have the unique property of overlapping ESC and MSC characteristics, as revealed by their markers; (3) UC-stem cells are not turnorigenic; and (4) they are hypoimmunogenic. Thus, it is believed that because UCMSCs are ideal for clinical allogeneic use in various regenerative therapies, this stem cell type is a potential candidate for a universal off-the-shelf HAR-MSC product. The expansion medium for UCMSCs contains Dulbecco's Modified Eagle Medium/F-12 (DMEM) enriched with 5% human platelet lysate obtained from healthy donors, 10% fetal bovine serum (FBS), 1×penicillin/streptomycin, 1×sodium pyruvate, 1×nonessential amino acids, and 500 IU heparin (Pharmatex). UCMSCs will be incubated in the MSC expansion medium at 37° C. in a humidified atmosphere with 5% CO2 (day 0). Forty percent of the medium will be changed every 3-4 days. After 2 weeks, the adherent UCMSCs will be detached (Passage 0), centrifuged at 1,200 rpm for 10 min, resuspended in the MSC expansion medium, and re-plated at a density of 100-200 cells/cm², and again cultured until full confluence is reached (Passage 1). At Passage 1, living cells (counted by trypan blue dye exclusion) are transduced with AAV-HAR. HAR-modified UCMSCs will then be expanded, following the procedure for the first passage but with the addition of puromycin to eliminate UCMSCs failing to express the HAR construct.

Generation of HAR-UCMSCs using AAVs

In preliminary studies, the transfection conditions were optimized of MSCs with a pDNA/Ip/HA ternary complex containing the Luciferase-expressing plasmid DNA vector (pGEM®-luc DNA (Promega, Fitchburg, WI), successfully generating HAR-MSCs using this non-viral vector. Using the same HAR construct, AAV-HAR will be custom produced by GeneCopoela (Rockville, MD). The AAV-HAR CXCR4-BMP2II/ALK3 construct contains the extracellular domain of CXCR4 (NCBI Reference#: NP_003458.1), CD8α hinge (NCBI Reference#: NP_001139345.1), CD8α transmembrane (NCBI Reference#: NP_001139345.1), BMP2R2 (NCBI Reference#: NP_001195.2), and ALK3 (NCBI Reference#: NP_004320.2) domains along with the EF-1α promotor. The AAV-HAR CXCR4-Blank construct contains the extracellular domain of CXCR4, the CD8α hinge, and the CD8α transmembrane domain with the EF-1α promotor. All pEZ-AV02 containing each HAR will be digested with BamHI/EcoRI and sub-cloned into the corresponding restriction sites of pAAV-IRES-Puro. pAAV-IRES-Puro-HAR's will be co-transfected with pAAV-DJ/8 Vector (containing AAV-2 Rep, AAV-DJ/8 Cap, and puromycin resistance genes) and pHelper Vector (containing Adeno E2A, Adeno E4, and Adeno VA) into the AAV-293 cell line, using Lipofectamine 2000. Each AAV-HAR vector will be purified using ViraBin AAV purification kits and the gene copy number will be assessed with QuickTiter AAV quantitation kits (Cell Biolab; San Diego, CA). when UCMSCs reach 70% confluence in T-75 flasks, cells will be infected with AAV-HAR (CXCR4-BMP2RII/ALK3), AAV-CXCR4 (negative control), or AAV-BMP2RII/ALK3 (negative control). In brief, UCMSCs are first plated in 6-well plates (5×10⁴ cells/well) and maintained for one day. The cells will then be washed twice and transfected by HAR-AAV particles with incremental multiplicity of infection (MOI) 50, 100, 250, 500, or 1000. After 24 hours, the HAR AAV-transduced cells will be maintained in the expansion medium containing 2 μg/ml of puromycin for 48 hours, then in UCMSC expansion medium without puromycin.

### Verification of Successful Transduction of HAR by AAV

UCMSCs transduced by the HAR-AAV vector will be characterized to verify the expression of CXCR4 on their cell surface and their binding of SDF-1, according to the procedures used in the preliminary studies. In addition, changes in mRNA and protein expressions following AAV-HAR transduction will be determined. Upon transfer of AAV-HAR into UCMSCs, the HAR (CXCR4-BMP2RII/ALK3) gene is transcribed into mRNA. There are two primary methods for detecting mRNA generated from the hybrid vector (versus mRNAs transcribed from the native genes): RNA-sequencing and RNAscope in situ hybridization. These methods allow us to determine the abundance and subcellular location, respectively, of the HAR-related mRNA. Quantitation of HAR-related mRNA is more relevant than detection of the HAR vector, since HAR-related mRNAs are translated into the functional HAR-induced protein. In addition, to detect HAR expression, the presence of CXCR4 on the cell surface will be determined by flow cytometry and confocal microscopy. HAR protein will also be assayed by immunoprecipitation (IP) or co-immunoprecipitation (co-IP) in cell lysates for quantitative analysis of HAR-expression, as well as its post-translational modifications and interaction with its down-stream partner molecules.

### Characterization of HAR-UCMSC

First, to evaluate the viability and growth of HAR-UCMSCs in comparison with native MSCs, cells will be stained with calcein on day 1, 4, 7, and 14 and visualized by fluorescence microscopy. Cell viabilities will be calculated by using the built-in function of NIH lmageJ software for the study of proliferation. To characterize cell spreading behavior, native MSCs, the two negative-control AAV-transduced MSCs, or AAV-HAR-transduced MSCs will be fixed with 4% paraformaldehyde on day 7, 14 or 21. Fixed cells will be stained with phalloidin and 4′,6-diamidino-2-phenylindole (DAPI) to visualize filamentous F-actin and cell nuclei, respectively. Whole cells will be visualized with an inverted microscope for phase contrast images and a confocal microscope for fluorescence images. Qualitative and quantitative data will be obtained from fluorescent staining and by colorimetric study.

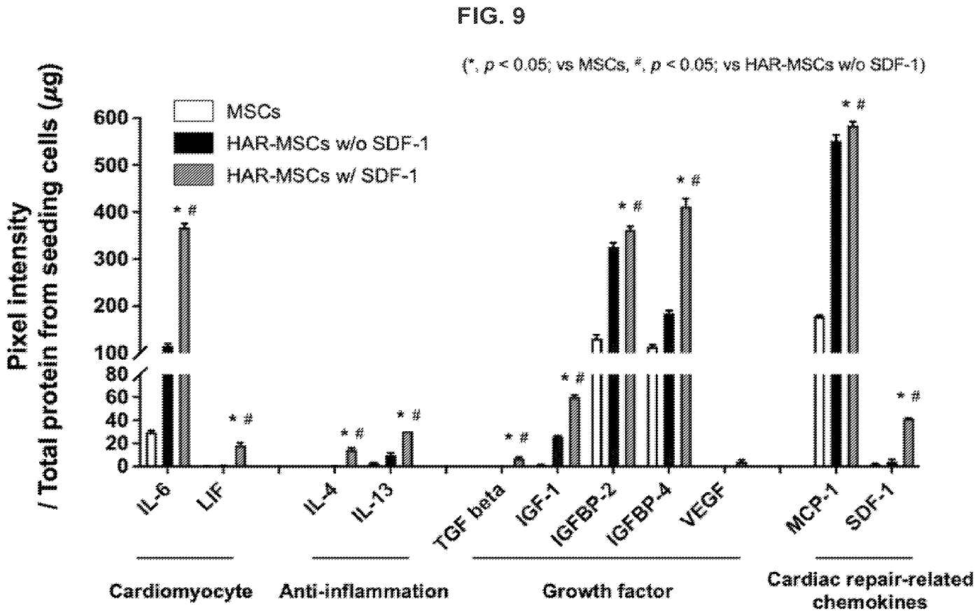

Cytokines secreted by cardiomyocyte-like MSCs differentiated from the HAR-UCMSCs may help prevent the progression of tissue damage caused by prolonged inflammation during LV remodeling after AMII. In a preliminary study, the cytokine secretion of cardiomyocyte-like MSCs was profiled in the absence or presence of SDF-1. Native MSCs, HAR-MSCs without SDF-1, and NAR-MSCs exposed to SDF-1 were cultured for 21 days and processed for a human cytokine antibody array (Human Cytokine Array C6, RayBiotech Inc) according to manufacturer's protocol. Immunoreactivity was detected using the ChemiDoc™ XRS+ detection system (BIORAD iNtRON Biotechnology). The signal density representing each protein was semi-quantitatively analyzed using ImageJ software and normalized to the total protein mass of each cell lysate. As shown in FIG. 9, increased production of cytokines associated with cardiogenic differentiation (IL-6 and LIF) and potential cardiac healing (anti-inflammatory, growth factor- and cardiac repair-related chemokines) was verified in the HAR-MSCs (CXCR4-BMP2RII/ALK3) exposed to SDF-1. This assay will be repeated in HAR-UCMSCs generated by the AAV methodology.

### Immunophenotypic Characterization of HAR-UCMSCs Compared to Native MSCs

To better understand phenotypic changes in HAR-UCMSCs, immunophenotypic characterization of cell surface markers and receptors will be performed in: native UCMSCs, the control AAV-transduced UCMSCs, and AAV-NAR-transduced UCMSCs, with or without exposure to SDF-1. Cells will be collected during expansion and washed with PBS followed by labeling with antibodies to detect UCMSCs-specific surface markers (CD90, CD105, CD73, CD34, HLA-DR, HLA-PerCP, CD29, or CD44), to detect cardiomyocyte makers (cTnT, cTnI, Nkx2.5, GATA4, CD172a, Connexin 43, or Cx43), and to verify the co-stimulatory domains (BMP2RII and ALK3) and their target, pSmad1/5. Labeled cells will be analyzed by flow cytometry. In addition, the expression of growth factor receptors, TGFβ receptor 2 (TGFβR2), Activin receptors 2A and B (ACVR2A, ACVR2B), BMP receptors 1B and 2A (BMPR1B, BMPR2A), Frizzled (FZD) receptors 3, 4, 5, and 7, IGF1 receptor (IGF1R) (R&D Systems), Patched receptor, and PTH/PTHrP receptor (Santa Cruz Biotechnology) will also be analyzed. Data from AAV-transduced cells will be compared with the results obtained from native MSCs.

### Transcriptome Profiling

To compare the comprehensive characteristics of native UCMSCs and HAR-UCMSCs at the transcriptome level, transcriptome profiling will be conducted. A library will be constructed using the SENSE mRNA-Seq Library Prep Kit (Lexogen) according to the manufacturer's instructions. Total RNA will be prepared and incubated with magnetic beads conjugated with oligo-dT followed by isolation of mRNAs, Library production will be initiated by the random hybridization of starter/stopper heterodimers to the poly(A) RNA that remains bound to the magnetic beads. These starter/stopper heterodimers contain IIlumina-compatible linker sequences. A single-tube reverse transcription and ligation reaction will be extended from the starter to the next hybridized heterodimer, where the nascent cDNA insert will be ligated to the stopper. Second strand synthesis will be performed to release the library from the beads; then the library is amplified. High-throughput sequencing will be performed as paired-end 100 sequencing using HiSeq 2000 (Illumina Inc.), RNA-Seq reads will be mapped using the TopHat software tool to obtain the alignment file. The alignment file will be used for assembling transcripts, estimating their abundances and detecting differential expression of genes or isoforms using Cufflinks. Gene classification is based on mining of BioCarta (www.biocarta.com), GenMAPP (www.genmapp.org), DAVID (david.abcc.ncifcrf.gov), and Medline (www.ncbi.nlm.nih.gov). The library preparation and RNA sequencing will be performed through the NGS services provided by Novogene. Based on the transcriptome profiles, differences between native UCMSCs and HAR-UCMSCs can be identified. Additionally, transcriptional differences in the responses to SDF-1 between native MSCs and HAR-MSCs will be revealed.

### Example 6

### Optimal Dose and Schedule for Systemic (IV) Infusion of HAR-MSCs

To successfully leverage the advantages of systemic infusion over direct injection, the optimal dose and schedule of administration must initially be determined. After first ascertaining the maximum tolerated dose (MTD), various schedules of administration will be compared. Additionally, functional assessments, based on echocardiographic data and the organ distributions of infused MSCs, will be compared between HAR-MSCs-infused and control groups.

### AMII Rat Model

The Sprague Dawley rat model of AMII, a well-established and highly reproducible rodent model of AMII, will be used for the animal studies described herein. Briefly, male Sprague Dawley rats are sedated, then anesthetized with isoflurane and intubated. Through a left thoracotomy, the heart is exposed by incising the pericardium, the left anterior descending artery (LAD) is identified and then ligated 1-2 mm from the anterior tip of the left atrial appendage. Successful ligation of the LAD is confirmed by visible blanching of the left ventricle (LV). The chest is closed in layers and residual intrathoracic air is evacuated. Sham-operative control animals will undergo thoracotomy with exposure of the heart, but without ligation of the LAD.

### Maximum Tolerated Dose (MTD) of HAR-MSCs

The safety and optimal dose of intravenously infused HAR-MSCs will first be evaluated in a dose escalation study in normal Sprague Dawley rats. A crude approximation of the desired number of stem cells to be infused is calculated as follows: the adult rat heart contains 7-10×10⁷ cardiomyocytes; the maximum number of cardiomyocytes possibly lost with a 40% infarction of the LV would thus be 2.1-3.0×10⁷. Consequently, in the dose escalation study, the initial cell count will be 1.0×10⁴, followed by 10-fold increases to a maximum dose of 1.0×10⁷ (n=10 rats per dose). Stem cells suspended in 200 μL PBS at the desired concentration are infused into the tail vein using a 1 mL syringe with a 26-gauge cannula. The control group receives 200 μL PBS only. Safety will be tracked at each dose by documenting all adverse events. Data from the normal rats will inform initial dosage choices in the AMII model.

In AMII rats, cells are intravenously infused thirty minutes after ligation of the LAD. An iterative approach to dose escalation will be employed. After the initial dose escalation study, the data will be reviewed to determine the range of doses for the next set. Additional data points are added at 0.5 log intervals bracketing the previous MTD above or below the dose determined to be the MTD on initial review. If necessary, the dose escalation scale will be expanded to embrace lower or higher cell numbers in order to identify with clarity the MTD.

Experimental groups: (1) vehicle and (2-5) HAR-MSCs, four doses (Group 1: 1.0×10⁴, Group 2: 1.0×10⁵, Group 3: 1.0×10⁸, Group 4: 1.0×10⁷). Each treatment will be administrated to the rats by IV infusion. A total of 65 rats (30% lethality, 13 rats per group) will be required to complete this study.

### Optimal Schedule for IV Infusions of HAR-MSCs

A general principle of cell therapy is that repetitive infusions confer cumulative therapeutic effects and therefore are substantially more effective than a single administration. Using the AMII model and the previously described procedure, the safety and efficacy of the MTD dose of modified HAR-MSCs in a single IV infusion will first be confirmed. Four groups will be compared: Group 1: Sham operation (no LAD ligation); Group 2: AMII—Vehicle (PBS) only; Group 3: AMII—naïve MSCs; Group 4: AMII HARs-MSCs. Next, to optimize the schedule of IV infusions of HARs-MSCs, three regimens each encompassing 3 weeks will be compared: (1) once a week; (2) twice a week, and (3) three times a week. These repeated injections will be made in the first week post operation as SDF-1 expression may last up to a week, even though the peak expression reaches 3 days after AMII. At the conclusion of the designated interval, cardiac echocardiographic data will be acquired and analyzed by a trained sonographer blinded to the groups. Following echocardiography, the rats will be sacrificed, and heart tissue retrieved for additional studies.

Experimental groups: Group 1: Sham, Group 2: AMII-induced vehicle, Group 3: AMII-induced native MSCs, Group 4: AMII-induced HAR-MSCs. This study aims to determine the effective IV infusion time of HAR-MSCs required for therapeutic efficacy of AMII. Each treatment will be administered by IV infusion. For this experiment, a total of 156 rats (30% lethality, 13 rats per group×4 groups×3 schedules of multi-IV infusion) will be required to complete this study.

### Echocardiography and Organ Distribution of HAR-MSCs

To evaluate functional status of the heart after AMII and IV infusion of engineered MSCs, echocardiography will be performed at 3, 6, and 9 weeks postoperatively in the experimental groups. Echocardiography serves two purposes: first, by documenting initial (at 3 weeks) hypokinesis of the anterior wall of the left ventricle, it confirms successful induction of myocardial infarction by LAD ligation in each subject rat; secondly, it will provide data on functional recovery of rats in the four study groups. In addition to the parameters of global ventricular function, e.g., cardiac output, ejection fraction, and stroke volume, particular attention will be paid to end-systolic and end-diastolic ventricular diameter, fractional shortening, and wall thickness. Speckle tracking-based strain imaging will also be applied to assess both regional and global left ventricular function. Myocardial microstructure will be assessed by in vivo image analysis code to determine the extent of regenerated cardiac muscle within the scar. All echocardiography data will be acquired and analyzed by a trained sonographer blinded to the groups.

Major organs including liver, lung, heart, kidney, spleen, and brain will be isolated and analyzed by SPECT Imaging prior to histological analysis. The isolated organs will be processed for immunohistochemistry. Histological analysis including Prussian blue staining, human nucleus staining for MSC detection, inflammation, immune cell invasion, and apoptosis will be used to determine the distribution of HAR-MSCs. The imaging studies will be performed in AMII rats. Each rat will be assigned to one of the following groups: (1) vehicle-treatment; (2) naive MSCs: and (3) HAR-MSCs. In order to reach statistical significance, 6 rats will be necessary for each group. With 30% lethality (8 rats per group), 24 rats will be required for the tracking/histology study.

### Example 7

### Therapeutic Efficacy of HAR-Engineered MSCs In Vivo

Having optimized delivery of HAR-MSCs and obtained initial data regarding the safety and efficacy of IV infusion in the AMII rat model, here the regenerative potential of the HAR-engineered MSCs will be examined in detail and the consequent morphologic and functional changes in the myocardium damaged by ischemia. Importantly, the results obtained with the schedule of IV infusions will be compared to the single intramyocardial injection.

### Study Groups of Rats

The experimental treatment groups are as follows: (1) Sham operation; (2) AMII—IV infused vehicle (PBS); (3) AMII—IV infused native MSCs; (4) AMII—IV infused HAR-MSCs; and (5) AMII—HARs-MSCs via a single intracardiac injection. For Groups 2, 3, 4, and 5, the infusions or injections are delivered 30 minutes after LAD ligation.

### Echocardiography, Histology, and Immunohistochemistry

To evaluate the functional status of the AMII hearts in a larger number of animals, echocardiography will be performed at 3, 6, and 9 weeks to confirm the extent of the initial ischemic injury and to evaluate functional recovery. Echocardiography will be performed as previously described. All echocardiography data will be acquired and analyzed by a trained sonographer blinded to the groups.

Experimental groups: Group 1: Sham, Group 2: AMII-induced vehicle, Group 3: AMII-induced native MSCs, Group 4: AMII-induced HAR-MSCs (G2 to G4 by IV infusion), and Group 5: AMII-induced HAR-MSCs by single intramyocardial injection. This study aims to investigate the in vivo cardiac functional improvement of HAR-MSCs required for therapeutic efficacy of AMII. For repeated cell administrations, a total of 130 rats (30% lethality, 26 rats×5 groups) will be required to complete this study.

Hearts will be obtained from the treatment groups at the conclusion of the designated interval of follow-up. After echocardiography and sacrifice, hearts will be harvested and fixed in 10% (v/v) formalin at room temperature (RT) for 48 hours, then stored in 70% (v/v) EtOH until sectioning. Paraffin-embedded slices are air-dried for 30 minutes at RT and in a dry 55-60° C. oven for an additional 30 minutes. The slides are de-paraffinized in three changes of xylene (5 minutes each), rehydrated in ethanol (100%×2, 95%×2, 70%×1, 1 min each), then placed in dH₂O. Staining of collagen with Masson's trichrome allows measurement of infarct size; H&E-stained sections are imaged to observe myocardial structures and immune cell infiltration. The infarcted region within the LV is quantified by outlining the collagen-stained area, the total LV epicardial area, and the total LV endocardial area in NIH ImageJ software, then calculating the infarcted area. Immunohistochemistry will also be performed to detect cardiac troponin T(cTnT), cardiac troponin I (cTnI), and sarcomere α-actinin (CMs),—all three are markers of mature cardiomyocytes—and fibroblast-specific protein 1 (Fsp-1), a fibroblast marker. Sections are incubated with the primary antibody for one hour at RT, followed by application of FITC- or Texas Red-conjugated secondary antibodies (Abcam). Signals of cTnT, cTnI, CMs and Fsp-1 will be quantified using NIH imageJ software.

### Quantitative Real-Time PCR (q RT-PCR)

Appropriate heart samples obtained at sacrifice will be used for qRT-PCR analysis. Briefly, total RNA is isolated with TRlzol reagent (lnvitrogen), according to the manufacturer's protocol, and mRNA is reverse-transcribed into complementary DNA (cDNA) using the iScript™ cDNA synthesis kit (Bio-rad). Quantitative PCR analysis is performed by iTaq™ universal SYBR® Green supermix (Bio-rad) with a CFX96™ real-time PCR detection system (Bio-rad), according to the manufacturer's protocols. Target gene expression is normalized to the glyceraldehyde-3-phosphate dehydrogenase (GAPDH) gene. For quantitative analysis, mature cardiomyocyte-related gene-specific primers have been designed to amplify NK2 Homeobox 5 (Nkx2.5), GATA binding protein 4 (GATA4), cTnT, cTnI and GAPDH. Primers pairs are shown in Table 4.

TABLE 4
Quantitative PCR Primer Pairs

| Gene | Forward primer (5′→3′) | SEQ ID | Reverse primer (5′→3′) | SED ID |
|---|---|---|---|---|
| Nkx2.5 | CACCGGCCAAGTGTGCGTCT | SEQ ID | GCAGCGCGCACAGCTCTTTC | SEQ ID |
| | | NO: 41 | | NO: 42 |
| GATA4 | CGGCGAGGAGGAAGGAGCCA | SEQ ID | TGGGGGCAGAAGACGGAGGG | SEQ ID |
| | | NO: 43 | | NO: 44 |
| cTnT | GGAGGAGAACAGGAGGAAGG | SEQ ID | CTGGATGTAACCCCCAAAATGC | SEQ ID |
| | | NO: 45 | | NO: 46 |
| cTnI | CGTGTGGACAAGGTGGATGA | SEQ ID | GCCGCTTAAACTTGCCTCG | SEQ ID |
| | | NO: 47 | | NO: 48 |
| GAPDH | ACCCACTCCTCCACCTTTGAC | SEQ ID | TGTTGCTGTAGCCAAATTCGTT | SEQ ID |
| | | NO: 49 | | NO: 50 |

All reactions are performed in a final reaction mixture (20 μL) containing 1×final concentration of SYBR supermix, 500 nmol/l of gene-specific primers, and 1 ml of template, and amplified under the following conditions: initial denaturation at 95° C. for 1 minute, followed by 45 cycles of 95° C. for 15 seconds, 56 CC for 15 seconds, and 72° C. for 15 seconds, with a final extension at 72° C. for 5 minutes. After amplification, the baseline and threshold levels for each reaction will be calculated by two methods: 1) the company's software package (Bio-rad); and 2) the comparative CT (ΔΔCT) method to calculate the relative expression of targeted genes. The qRT-PCR will be performed in triplicate with spiking of one of the reaction tubes with a low amount of DNA.

### CXCR4 and SDF-1 Null Mouse AMII Model

Over-expression of SDF-1 in the heart begins at 1 hour after the injury, continues to express SDF-1 for 3 days, and returns to the baseline thereafter. During this process, CXCR4 is also up-regulated in cardiomyocytes for around 36 to 48 hours after MI, and these cardiomyocytes expressing CXCR4 can competitively respond to the SDF-1 secreted from the acutely infarcted myocardium. This tissue SDF-1/CXCR4 binding may inhibit the migration of exogenously infused HAR-MSCs CXCR4/BMP2RII-ALK3. Moreover, endogenous MSCs express CXCR4 in response to SDF-1 secreted from the ischemic myocardium. Blocking the homing of endogenous CXCR4-expressing MSCs toward the ischemic heart is required for the precise determination of the effects of the infused HAR-MSCs in the animal models. Since there are no commercially available CXCR4-null rats, CXCR4-null mice (B6.129X-Cxcr4tm1Qma/J) will be used to subtract the possible artifact caused by the endogenous MSC horning mechanism. Four breeding colonies, each housing two females and one male, will be maintained.

This study allows demonstration of the sole effect of the homing of the exogenously transferred HAR-MSCs on cardiac repair. AMII models will be generated with the CXCR4-null mice by means of the previously described procedure developed for rat AMII models. CXCR4-null mouse models will be systemically injected with HAR-MSCs (the number of MSCs infused will be determined by the number of MSCs/kg used in the rat models). Appropriate functional assessments will be performed in this mouse model. Major organs will be isolated after 3 days, minced, and digested to single-cell suspensions for flow cytometry and then processed for immunohistochemistry. Collected cells will be analyzed with appropriate antibodies. Additionally, the experiments will be repeated with SDF-1 (CXCL12) gene-flanked null mice (B6(FVB)-Cxcl12tm1.1Link/J) in order to cross-verify the efficacy of HAR-MSCs CXCR4/BMP2RII-ALK3.

Each mouse will be assigned to one of the following groups: (1) vehicle, (2) native MSCs, (3) HAR-MSCs CXCR4-BMP2RII-ALK4, (4) non-treated control, (5) thoracotomy, or (6) non-AMII. In order to reach statistical significance, 8 mice will be necessary for each treatment group. With 30% lethality (11 mice per group), a total of 66 B6.129X-Cxcr4tm1Qma/J mice and 66 B6(FVB)-Cxcl12tm1.1Link/J mice, will be required to complete this study.

### Angiogenesis and Neovascularization in the Myocardial Ischemic Zone

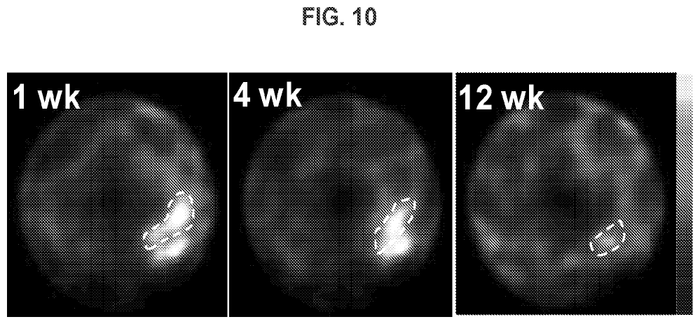

Angiogenesis and neovascularization are well known processes underlying the cardiac repair by stem cells. To assess angiogenesis and neovascularization, 99mTc-3P-RGD2, a radiolabeled peptide dimer is used to detect integrin αvβ3. The images in FIG. 10 demonstrate the robust angiogenesis in the myocardium 1-4 weeks post-infarction, an example of how the technique might be applied to visualizing recovery from infarction. The high-resolution SPECT imaging technology can likely be utilized to track IV infused MSCs labeled with a radiotracer in order to detect and measure their accumulation in the ischemic heart. To verify successful infarction after ligation of the LAD, animals will be imaged 24 hours postoperatively with 99mTc-duramycin; a cell death biomarker targeting externalized phosphatidylethanolamine. Two hours after the intravenous injection of radiotracer; images are acquired over a 10 to 15-minute interval. If no hot-spot indicating uptake of 99mTc-duramycin is detected, that animal will be excluded. At 3, 6, and 9 weeks post-operatively, correlations between the uptake of 99mTc-duramycin (cell death) and 99mTc-3P-RGD2 (angiogenesis), with or without infusion of MAR-MSCs, will be evaluated. This study will provide data about temporal changes in the extent of myocardial infarction, in LV remodeling, and in the progression of neovascularization.

Experimental groups: Group 1: Sham, Group 2: AMII-induced vehicle, Group 3: AMII-induced naive MSCs, Group 4: AMII-induced MAR-MSCs (G2 to G4 by IV infusion), and Group 5: AMII-induced HAR-MSCs by single intramyocardial injection. To reach statistical significance, 5 rats per heart excision will be required. With 30% lethality (7 rats per heart excision), a total of 105 rats (7 rats×3-times, five groups) will be necessary to complete this study.

### Tracking Systemically Infused HAR-MSCs in Vivo

For in vivo tracking of cells delivered by IV infusion (or directly injected into the myocardium), native MSCs and HAR-MSCs are first labeled with micron-sized iron oxide particle (MPIO) microspheres (product No. MC03F19781, Bangs Laboratories). The MPIOs, which serve as both an MRI contrast agent and a histological marker; measure 0.9 μm in diameter and are composed of polystyrene with 62% (w/w) iron oxide: they are also labeled with a red fluorescent dye (Flash Red). To determine the optimal conditions for labeling MSCs, decreasing amounts of MPIOs are used, ranging from 1.8×10⁸ to 0.7×10⁶ MPIOs per cm² of cell culture surface area (corresponding to 100 μL to 0.4 μL MPIO suspension per ml medium). The infused MSCs are imaged in vivo using a clinical 3.0 T MRI scanner (80 mT/m max strength, slew rate: 200 mT×ms/m, Intera Achieva, Phillips Medical Systems). To enhance signal-to-noise-ratio, the MRI device will be equipped with a dedicated experimental small animal solenoid coil (Phillips Medical Systems). The rats will be anesthetized with 1.25% isoflurane (1 L/min O₂).

For electrocardiographic (ECG) gating, MRI-compatible pediatric ECG electrodes (blue sensor BR/BRS, Ambu A/S) will be attached to the animals' paws. Serial MRI scans of transplanted hearts will be performed weekly for 3, 6, and 9 weeks beginning 3 weeks postoperatively. Long-axis images of the left ventricle will be obtained by ECG-gated sagittal scans of six slices with six cardiac phases to localize the MPIO-labeled cells. Infused MSCs within the ventricular wall are visualized as signal voids (not present in sham operated animals). Image files are saved with the visualization software DICOM viewer R2.5 v1.1 (Philips).

Transverse MRI images covering the entire left ventricle are used to map endocardial and epicardial contours. Left ventricular end-systolic (LVES) and end-diastolic (LVED) volumes are calculated using image analysis software Segment v1.8 (Medviso). Left ventricular ejection fraction (LVEF) is calculated from LVES and LVED volumes. Data will be analyzed independently by three experienced researchers. Sagittal images showing signal voids in the left ventricular walls are analyzed for signal void areas and intensities by ImageJ software v1.42q (rsbweb.nih.gov/ij). The threshold for specific signal void intensity will be defined as less than 50% of background; background intensities are derived from images of the septum. Each slice will be scanned for areas with signal intensities below the threshold level. Signal voids in the left ventricular wall will then be quantified for area size and mean signal intensity. It is anticipated that the data will support the hypothesis that IV-infused HAR-MSCs target and repopulate the myocardium damaged by ischemic. If the image is not clear enough to recognize transplanted cells in vivo by current MPIO-labeled imaging procedures, then the concentration of MPIO microspheres used for labeling will be increased. An alternative approach is to co-label MSCs containing MPIOs with the fluorescent vital cell stain Vybrant 1,1-dioctadecyl-3,3,3′,3′-tetramethylindocarbocyanine perchlorate (DiI; Invitrogen).

Experimental groups: Group 1: Sham operated, Group 2: AMII plus vehicle, Group 3: AMII plus naïve MSCs, Group 4: AMII plus HAR-MSCs, all by IV infusion); and Group 5: AMII plus HAR-MSCs delivered by a single intramyocardial injection. For repeated cell administrations, a total of 65 rats (13 rats (30% lethality, 13 rats per group×5 groups) will be required to complete this study.

What is claimed:

1. An engineered stem cell comprising a nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain.

2. The stem cell of claim 1, wherein the HAR further comprises cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain.

3. The stem cell of claim 1, wherein the stem cell is an embryonic stem cell, perinatal stem cell, adult stem cell, induced pluripotent stem cell, tissue-specific stem cell, mesenchymal stem cell, hematopoietic stem cell, mesenchymal stem cell, neural stem cell, or epithelial stem cell.

4. The stem cell of claim 1, wherein the stem cell is a mesenchymal stem cell.

5. The stem cell of claim 1, wherein the stem cell is a subject-derived stem cell.

6. The stem cell of claim 1, wherein the stem cell is a mouse, rat, rabbit, pig, or human mesenchymal stem cell.

7. The stem cell of claim 1, wherein the CXCR domain comprises a CXCR4 domain.

8. The stem cell of claim 1, wherein the CXCR domain is encoded by a nucleic acid at least 90% identical to SEQ ID NO: 9.

9. The stem cell of claim 1, wherein the CXCR domain comprises a polypeptide is at least 90% identical to SEQ ID NO: 10.

10. The stem cell of claim 1, wherein the nucleic acid is at least 90% identical to SEQ ID NO: 1.

11. The stem cell of claim 1, wherein the HAR the polypeptide is at least 90% identical to SEQ ID NO: 2.

12. The stem cell of claim 1, wherein the nucleic acid encoding the HAR polypeptide is comprised in an extra chromosomal vector.

13. The stem cell of claim 1, wherein the nucleic acid encoding the HAR polypeptide is integrated into the genome of the cell.

14. The stem cell of claim 1, wherein expression of the HAR polypeptide is driven by a promotor.

15. A method or means for treating cardiovascular disease or disorder in a subject in need thereof, the method comprising contacting cardiovascular tissue with an effective amount of one or more of the engineered stem cells of claim 1.

16. The method of claim 15, wherein the cardiovascular disease or disorder comprises coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions.

17. The method of claim 15, wherein the cardiovascular disease or disorder is myocardial infarction.

18. Use of an engineered stem cell of claim 1 for treating or preventing a cardiovascular disease or disorder comprising coronary heart disease, coronary artery disease, acute coronary syndrome, cardiomyopathy, myocardial infarction, angina pectoris, ischemic cardiomyopathy, rheumatic heart disease, congestive heart failure, aorta disease, heart valve disease, pericardial disease, congenital heart disease, abnormal heart rhythms or arrhythmias, atherosclerosis, restenosis, ischemic stroke, cerebrovascular disease, peripheral vascular disease, vascular inflammation, vascular autoimmune diseases, Marfan syndrome, deep vein thrombosis, pulmonary embolism, or other coronary or vascular conditions.

19. The use of claim 18, wherein the cardiovascular disease or disorder is myocardial infarction.

20. A nucleic acid encoding a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain.

21. The nucleic acid of claim 20, wherein the HAR further comprises a cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain.

22. The nucleic acid of claim 20, wherein the nucleic acid is at east 90% identical to SEQ ID NO: 1 or the complement thereof.

23. The nucleic acid of claim 20, wherein the nucleic acid encodes a HAR polypeptide at least 90% identical to SEQ ID NO: 2.

24. An isolated a hybrid allosteric receptor (HAR) polypeptide comprising an extracellular C-X-C chemokine receptor (CXCR) domain, an intracellular bone morphogenetic protein 2 type II receptor (BMP2RII) domain, and an intracellular bone morphogenetic protein (BMP) type I receptor (ALK3) domain.

25. The isolated HAR of claim 24, wherein the HAR further comprises a cluster of differentiation 8 alpha (CD8α) leader sequence, a CD8α hinge domain, a CD8α transmembrane domain between the CXCR domain and the BMPR2II domain, and a polyglycine linker between the BMPR2II domain and the ALK3 domain.

26. The isolated HAR of claim 24, wherein the polypeptide is encoded by a nucleic acid at least 90% identical to SEQ ID NO: 1.

27. The isolated HAR of claim 24, wherein the HAR polypeptide is at least 90% identical to SEQ ID NO: 2.

28. A method for manufacturing an engineered stem cell, the method comprising:
(a) preparing a nucleic acid encoding a polypeptide comprising an extracellular C-X-C chemokine receptor (CXCR) domain, a cluster of differentiation 8 alpha (CD8α) hinge domain, a CD8α transmembrane domain, an intracellular bone morphogenetic protein 2 type II receptor (BMP2RII) domain, and an intracellular bone morphogenetic protein (BMP) type I receptor (ALK3) domain;
(b) introducing the nucleic acid of (a) into a stem cell; and
(c) isolating the transfected stem cells comprising the introduced nucleic acid.

29. The method of claim 28, wherein the nucleic acid is at east 90% identical to SEQ ID NO: 1.

30. The method of claim 28, wherein the stem cell is a mesenchymal stem cell.

31. The method of claim 28, wherein the stem cell expresses a HAR polypeptide.

32. An engineered stem cell produced by the method of claim 28.

33. The stem cell of claim 31, wherein the stem cell is a mesenchymal stem cell.

34. An engineered stem cell comprising a nucleic acid encoding a hybrid allosteric receptor (HAR) polypeptide comprising an extracellular receptor domain, and one or more intracellular domains.

35. The stem cell of claim 34, wherein the extracellular domain and one or more intracellular domains are each selected from Chemokine (C-C motif) receptor 1 (CCR1, Chemokine (C-C motif) receptor 2 (CCR2), Chemokine (C-C motif) receptor 3 (CCR3), Chemokine (C-C motif) receptor 4 (CCR4), Chemokine (C-C motif) receptor 5 (CCR5), Chemokine (C-C motif) receptor 6 (CCR6), Chemokine (C-C motif) receptor 7 (CCR7), Chemokine (C-C motif) receptor 8 (CCR8), Chemokine (C-C motif) receptor 9 (CCR9), Chemokine (C-C motif) receptor 10 (CCR10), Chemokine (C-X-C motif) receptor 1 (CXCR1), Chemokine (C-X-C motif) receptor 2 (CXCR2), Chemokine (C-X-C motif) receptor 3 (CXCR3), Chemokine (C-X-C motif) receptor 4 (CXCR4), Chemokine (C-X-C motif) receptor 5 (CXCR5), Chemokine (C-X-C motif) receptor 6 (CXCR6), C-X3-C motif chemokine receptor 1 (CX3CR1), Chemokine (C motif) XC receptor 1 (XCR1), Atypical chemokine receptor 1 (ACKR1), Atypical chemokine receptor 2 (ACKR2), Atypical chemokine receptor 3 (ACKR3), Atypical chemokine receptor 4 (ACKR4), C-C Motif Chemokine Receptor Like 2 (CCRL2), Activin A Receptor Type 1 (ACVR1), Bone morphogenetic protein receptor type 1A (BMPRIA(ALK3)), Bone morphogenetic protein receptor type 1B (BMPRIB(ALK6)), Bone morphogenetic protein receptor type 2 (BMPR2), Activin A receptor type 2A (ACVR2A), Activin A receptor type 2B (ACVR2B), Transforming growth factor beta receptor 1 (TGFBR1), Transforming growth factor beta receptor 2 (TGFBR2), Vascular endothelial growth factor receptor 1 (VEGFR1), Vascular endothelial growth factor receptor 2 (VEGFR2), Vascular endothelial growth factor receptor 3 (VEGFR3), Epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), Platelet-derived growth factor receptor A (PDGFRα), Platelet-derived growth factor receptor B (PDGFRβ), Fibroblast growth factor receptor 1 (FGFR1), Fibroblast growth factor receptor 2 (FGFR2), Fibroblast growth factor receptor 3 (FGFR3), Fibroblast growth factor receptor 4 (FGFR4), Hepatocyte growth factor (HGF), Tropomyosin-related kinase A (TrkA), Tropomyosin-related kinase B (TrkB), Tropomyosin-related kinase C (TrkC), p75 neurotrophin receptor (p75NTR), Erythropoietin receptor (EPOR), Growth hormone receptor (GHR), Prolactin receptor (PRLR), Thrombopoietin receptor (TOPR), Granulocyte colony-stimulating factor receptor (GCSFR), Leukemia inhibitory factor receptor (LIFR), Ciliary neurotrophic factor receptor (CNTER), Cardiotrophin-like cytokine factor 1 (CLCF1), Oncostatin M receptor (OSMR), Interferon alpha receptor 1 (IFNAR1), Interferon alpha receptor 2 (IFNAR2), Glycoprotein 130 (gp130), Glycoprotein 140 (gp140), Integrin subunit alpha 1 (ITGA1), Integrin subunit alpha 2 (ITGA2), Integrin subunit alpha 3 (ITGA3), Integrin subunit alpha 4 (ITGA4), Integrin subunit alpha 5 (ITGA5), Integrin subunit alpha 6 (ITGA6), Integrin subunit alpha 7 (ITGA7), Integrin subunit alpha L (ITGAL), Integrin subunit alpha M (ITGAM), Integrin subunit alpha IIB (ITGAIIB), Integrin subunit alpha V (ITGAV), Integrin subunit beta 1 (ITGB1), Integrin subunit beta 2 (ITGB2), Integrin subunit beta 3 (ITGB3), Integrin subunit beta 4 (ITGB4), Integrin subunit beta 5 (ITGB5), Integrin subunit beta 6 (ITGB6), Integrin subunit beta 8 (ITGB8), Interleukin 1 receptor type 1 (IL1R1), Interleukin 1 receptor type 2 (IL1R2), Interleukin 2 receptor alpha (IL2RA), Interleukin 2 receptor beta (IL2RB), Interleukin 2 receptor gamma (IL2RG), Interleukin 3 receptor alpha (IL3RA), Interleukin 4 receptor (IL4R), Interleukin 5 receptor alpha (IL5RA), Interleukin 6 receptor (IL6R), Interleukin 7 receptor (IL7R), Interleukin 9 receptor (IL9R), Interleukin 11 receptor alpha (IL11RA), Interleukin 12 receptor beta 1 (IL12RB1), Interleukin 13 receptor alpha 1 (IL13RA1), Interleukin 13 receptor alpha 2 (IL13RA2), Interleukin 15 receptor (IL15R), Interleukin 18 receptor 1 (IL18R1), Interleukin 21 receptor (IL21R), Interleukin 23 receptor (IL23R), Interleukin 27 receptor alpha (IL27RA), Interleukin 10 receptor alpha (IL10RA), Interleukin 10 receptor beta (IL10RB), Interleukin 20 receptor alpha (IL20RA), Interleukin 20 receptor beta (IL20RB), Interleukin 22 receptor alpha 1 (IL22RA1), Interleukin 22 receptor alpha 2 (IL22RA2), Interleukin 28 receptor alpha (IL28RA), or Interleukin 28 receptor beta (IL28RB).

36. The stem cell of claim 34, wherein the HAR comprises one intracellular domain and two intracellular domains.

37. The stem cell of claim 34, wherein the HAR further comprises a leader sequence, a hinge domain, a transmembrane domain between the extracellular domain and the first intracellular domain, and a linker between the two intracellular domains.

38. The stem cell of claim 34, wherein the HAR comprises any of the domains listed in Table 3.
